(19) United States
(12) Patent Application Publication
(10) Pub. No.: US 2007/0066682 A1
(43) Pub. Date: Mar. 22, 2007

(54) Arylalkylamine vanadium (V) salts for the treatment and/or prevention of Diabetes mellitus

(76) Inventors: Miriam Royo Exposito, Barcelona (ES); Luc Marti Clauzel, Barcelona (ES); Anna Abella Martin, Sarit Joan Despi (ES); Silvia Garcia Vicente, Sant Feliu de Lobregat (ES); Xavier Testar Yinbert, Barcelona (ES); Antonio Zorzano Olarte, Barcelona (ES); Manuel Palacin Prieto, Barcelona (ES); Fernando Albericio Palomera, Barcelona (ES); Francesc Yraola Font, Barcelona (ES); Alec Mian, Barcelona (ES)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/428,534

(22) Filed: Jul. 3, 2006

Related U.S. Application Data
(60) Provisional application No. 60/800,058, filed on May 12, 2006.
(60) Provisional application No. 60/695,943, filed on Jul. 1, 2005.

(51) Int. Cl. A61K 31/28; C07F 9/00

(52) U.S. Cl. 514/492; 556/42

(57) ABSTRACT

This invention provides compounds of formula (IIA) 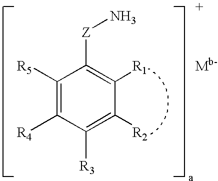
and pharmaceutical compositions thereof, where M, a, b, and R₁-R₅ are as defined herein, for treating human type 1 and type 2 diabetes, particularly insulin-resistant diabetes. Pharmaceutical compositions comprising the compounds of formula (IIA) are also disclosed.

This application claims priority to U.S. provisional application Ser. No. 60/695,493, filed Jul. 1, 2005, and Ser. No. 60/800,058, filed May 12, 2006, the disclosures of each of which are explicitly incorporated by reference herein.

### BACKGROUND OF THE INVENTION

1. Field of the Invention

This invention relates to diabetes and compounds and pharmaceutical compositions for treating diabetes. In particular, this invention relates to methods, compounds and pharmaceutical compositions for treating human type 1 and type 2 diabetes. Specifically, the invention provides compounds that can substitute for insulin in vivo and methods for using pharmaceutical compositions of such compounds for treating human type 1 and type 2 diabetes. In particular, this invention relates to salts of vanadium (V) with arylalkylamines for use as insulin mimickers, i.e. compounds that can produce insulin-like effects in the absence of insulin.

2. Background of the Related Art

Diabetes, especially in its most common form Diabetes mellitus, is a major global health problem that is recognized by the World Health Organization to be reaching epidemic proportions. It is now the fourth leading cause of death in most developed countries and a disease that is increasing rapidly in countries undergoing industrialization.

Diabetes mellitus is a metabolic disorder in which the ability to oxidize carbohydrates is practically lost, usually due to faulty pancreatic activity, especially of the islets of Langerhans, and consequent disturbance of normal insulin mechanism. It is characterized by abnormally elevated glucose levels in the plasma and urine, by excessive urine excretion and by episodic ketoacidosis. Additional symptoms of diabetes mellitus include excessive thirst, glucosuria, polyuria, lipidema and hunger. If left untreated the disease can lead to fatal ketoacidosis. Diabetes mellitus can eventually damage the eyes, kidneys, heart and limbs and can endanger pregnancy. Clinical criteria that establish an individual as suffering from diabetes mellitus include fasting plasma glucose levels in excess of 126 mg/dl (7 mmol/L; normal levels are typically less than 100 mg/dl (<5.6 mmol/L)). Alternatively, patients may show a plasma glucose levels in excess of 200 mg/dL (11 mmol/L) at two times points during a glucose tolerance test (GTT), one of which must be within 2 hrs of ingestion of glucose.

Diabetes mellitus is usually classified into two major types, type 1 diabetes and type 2 diabetes. Type 1 diabetes, or insulin-dependent Diabetes mellitus (IDDM), is defined by development of ketoacidosis in the absence of insulin therapy. Type 1 diabetes most often manifests in childhood and is therefore also called juvenile onset diabetes. Rapid in onset and progress, it accounts for about 10 to 15 percent of all cases. Type 2 diabetes, or non-insulin-dependent Diabetes mellitus (NIDDM), is characterized by persistent hyperglycemia but rarely by ketoacidosis. Type 2 diabetes typically manifests after age 40 and progresses slowly. Due to its late onset, it has formerly been called adult-onset diabetes. Type 2 diabetes, which is by far the most frequently occurring type of diabetes, is often not accompanied by clinical illness in its initial stages and is detected instead by elevated blood or urine glucose levels.

Two major forms of type 2 diabetes are to be distinguished in the basis of their association (or not) with obesity. Of the two, the form associated with obesity is of increasing importance. Type 2 diabetes associated with obesity is presently developing at an epidemic rate and is thus of major interest. For example, in the United States the proportion of the population under 40 that can be clinically defined as obese now exceeds 25%. Even many children are obese and are developing type 2 diabetes at an alarming rate.

Diabetes type 1 and 2 are both now considered as a group of disorders with multiple causes, rather than a single disorder. Common to diabetes type 1 and 2 is that entry of glucose into cells is impaired. Entry of glucose into cells is typically catalyzed by insulin, a hormone secreted by Langerhans cells in the pancreas. By facilitating entry of sugar glucose into tissue cells of the body insulin provides energy for metabolic activities. Impairment of glucose uptake may be a result either of a deficiency in the amount of insulin produced in the body or of altered target cells not enabling the cells to take up glucose. Impairment of glucose uptake results in excess glucose build-up in the blood and excreted in the urine.

Insulin elicits anabolic and anti-catabolic responses by activation of several intracellular signalling pathways. The actions of insulin are initiated by its binding to the insulin receptor, which leads to the activation of the receptor's intrinsic tyrosine kinase (Hubbard et al., 1994, Nature 372: 746-754; Hubbard, 1997, EMBO J. 16: 5572-5581). The function of the receptor tyrosine kinase is essential for the biological effects of insulin (Hubbard et al., 1994, Id.; Hubbard, 1997, Id.; Ebina et al., 1985, Cell 40: 747-758; Ullrich et al., 1985, Nature 313: 756-761; White & Kahn, 1994, J. Biol. Chem. 269: 1-4). Insulin receptors phosphorylate several immediate substrates including insulin receptor substrate (IRS) proteins (White & Kahn, 1994, Id.). These events lead to the activation of downstream signalling molecules such as phosphatidylinositol 3-kinase, protein kinase B or atypical forms of protein kinase C.

The etiology of type 1 diabetes almost always includes a severe or total reduction in insulin production. This reduction is typically the result of an autoimmune destruction of beta-cells in the pancreas that are responsible for producing insulin. The most common therapy for insulin dependent Diabetes mellitus (type 1 diabetes) is the provision of insulin by injection, thereby replacing the deficiency.

Type 2 diabetes can result from genetic defects that cause both insulin resistance and insulin deficiency. In type 2 diabetes, the pancreas often produces a considerable quantity of insulin, whereas the hormone is unable to promote the utilization of glucose by tissues. In fact, a hallmark of type 2 diabetes is insulin resistance. A subset of diabetic patients showed severe insulin resistance and they require more than 2 U of insulin per kg and day (Tritos & Mantzoros, 1998, J. Clin. Endocrinol. Metab. 83: 3025-3030; Vestergaard et al., 2001, J. Intern. Med. 250: 406-414. The molecular basis for insulin resistance in type 2 diabetes remains poorly understood, however. Several studies have shown that obesity or type 2 diabetes are characterized by modest decreases in insulin receptor number (Olefsky et al., 1985, Amer. J. Med. 79: 12-22), reduction in insulin-stimulated receptor tyrosine kinase activity and defects in receptor-mediated IRS phosphorylation or phosphatidylinositol 3-kinase or protein kinase C-□ activation (Olefsky et al., 1985, Id; Beeson et al., 2003, Diabetes 52: 1926-1934; Caro et al., 1987, J. Clin. Invest 79: 1330-1337; Goodyear et al., 1995, J. Clin. Invest 95: 2195-2204; Kim et al., 1999, J. Clin. Invest 104: 733-741). Thus, at least a subset of type 2 diabetic patients have clear defects in insulin signalling that could be overcome by treatment aimed at augmenting the insulin signalling cascade, inter alia, by providing an insulin replacement that bypasses the insulin receptor.

Various efforts have been made to treat diabetes and in particular insulin resistant diabetes type 2. One such way of curing these conditions is to provide so called “insulin mimetics”, i.e. compounds capable of “mimicking” the functions of insulin such as to enable cells to take up glucose.

Several inorganic compounds have been reported to mimic the effects of insulin, in vivo as well as in isolated cells and tissues. Such mimetics include vanadium (IV)/(V) compounds. (Heyliger et al., 1985, Science 227: 1474-7); selenates (McNeill et al., 1991, Diabetes 40: 1675-8), lithium salts (Rodriquez-Gil et al., 1993, Arch. Biochem. Biophys. 301: 411-5), tungsten (VI) and molybdenum (VI) compounds (U.S. Pat. No. 5,595,763 and Li et al., 1995, Biochemistry 34: 6218-6225)).

Among the above inorganic compounds, vanadium and its derivatives have been proven as potent insulin-mimetics. There is convincing evidence for the effects of vanadates and peroxovanadium complexes (vanadium in its +5 oxidation state combined with oxygen, in particular orthovandate VO₄³⁻, see U.S. Pat. No. 4,882,171), and vanadyl VO²⁺ salts and complexes (vanadium in its +4 oxidation state; see U.S. Pat. No. 5,300,496) to increase cells' susceptibility for glucose uptake. Vanadium compounds are currently undergoing clinical trials in Europe and America. However, even though promising results for the transport of glucose into cells have been gathered, administration of vanadium compounds is accompanied by serious toxicity problems at effective doses. Administered concentrations must be close to toxic levels, if desired insulin-mimetic effects in animals are to be achieved. Considerable side effects are observed for vanadium-treatment that are independent from the chemical nature of the specific vanadium used for therapy (Domingo et al., 1991, Toxicology 66: 279-87.). Serious problems with vanadium compounds toxicity are observed at any kind of dosage suitable for lowering blood glucose levels, including a significant mortality rate.

Transport of glucose into cells in vivo has been mediated by using vanadate in combination with substrates of semicarbazide-sensitive amine-oxidase (SSAO), such as benzylamine or tyramine. As reported by Enrique-Tarancon et al. (1998, J. Biol. Chem. 273: 8025-8032 and Enrique-Tarancon et al., 2000, Biochem. J. 350: 171-180 or WO 02/38152), a combination of amines such as benzylamine or tyramine with vanadate was found to stimulate glucose transport and GLUT4 translocation in rat 3T3-L1 adipocytes. According to Enrique-Tarancon et al. (1998, 2000, supra) glucose transport is stimulated by an increase of GLUT4 carrier concentration on the cell surface, resulting from potent tyrosine phosphorylation. Similarly, Marti et al. (1998, J. Pharmacol. Exper. Therap. 285: 342-349) reported that glucose transport was stimulated using a combination of tyramine and vanadate. According to Marti et al. (1998, supra) stimulation of glucose transport was sensitive to MAO (monoamine oxidase) and SSAO inhibitors and to catalase. Marti et al. (1995, supra) also disclosed the use of vanadate in combination with tyramine.

Patent application WO 02/38152 A1 describes a pharmaceutical combination formed by vanadium (IV)/(V) compounds and amines of the semicarbazide-sensitive amine oxidase substrates group, which is potently synergic in producing an insulin effect. More recently, in vivo studies have also demonstrated the anti-diabetic properties of the combination of benzylamine or other arylalkylamines with vanadium in experimental models of type 1 and type 2 diabetes. (Marti, et al. Diabetes. 2003, 50(9), 2061-8; Abella, et al., Journal of Physiology and Biochemistry. 2003, 59(3), 153-60.) Thus, this combination is useful at low concentrations of the metal. However, these successes are tempered with the need to establish even the lowest possible effective doses for vanadate in order to avoid negative side effects of treatment due to toxicity of vanadate. Because the bifunctional protein of SSAO and vascular adhesion protein-1 (SSAO/VAP-1) is highly expressed in adipocytes, substrates that bind to SSAO are desirable.

Despite all the research efforts of the past, the treatment and/or prevention of Diabetes mellitus are far from being satisfactory. Therefore, it is useful to find new antidiabetic drugs, especially of the single ingredient kind. This is so because (generally) administration of a two-ingredient drug is less satisfactory than administration of a single ingredient one, from the dosage and simplicity points of view.

For the subset of diabetic patients showing severe insulin resistance who require more than 2 U of insulin per kg and day, therapy with insulin replacement compounds that bypass the insulin receptor may be an efficient strategy. In addition, since patients with type 1 diabetes depend on parenteral exogenous insulin injections for metabolic control, the discovery of orally active compounds that mimic insulin's effects could lead to alternative therapies for this disorder.

Thus there is a need in the art for compounds and pharmaceutical compositions that mimic the effects of insulin, or preferably, are insulin replacement compounds that act, for example, in the insulin signalling cascade at a point downstream from the insulin receptor, thereby overcoming severe insulin resistance caused, inter alia, by diminution of insulin receptor molecules at the cell surface.

### SUMMARY OF THE INVENTION

This invention provides compounds and pharmaceutical compositions thereof that are insulin mimetic or insulin replacement compounds that overcome severe insulin resistance caused, inter alia, by diminution of insulin receptor molecules at the cell surface. The invention provides compounds and pharmaceutical compositions thereof that are effective insulin mimetic or insulin replacement agents for treating and/or preventing diabetes, particularly for diabetes type II, that are effective at low doses and have minimal toxic side effects. Methods for using the compounds provided by the invention to formulate effective pharmaceutical compositions, and methods for administering said pharmaceutical compositions of the invention to an animal, most preferably a human, having diabetes, most preferably human type I or type II diabetes, are also provided.

In particular, this invention provides vanadate salts of arylalkylamines as antidiabetic agents, insulin sensitizers, insulin mimetic or insulin replacement compounds, which can be administered as a single ingredient, and which are even better insulin mimickers than combination of vanadium plus amine. Thus, an aspect of the present invention provides compounds of formula IIA (where M is preferably, but not necessarily, a vanadium ion or complex): 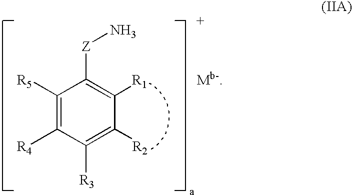
or pharmaceutically acceptable solvates or salts thereof, including hydrates,
wherein: Z is —(C₁-C₆ alkoxy)- or —(C₁-C₆ alkyl)-;
R₁, R₂, R₃, R₄ and R₅ are independently H, OH, (C₁-C₆)-alkyl, (C₁-C₆)-alkoxy, NR₆R₇, (CH₂)ₚNR₈R₉, O(CH₂)_{q}phenyl, CONR₁₀R₁₁, COR₁₂, CF₃, OCF₃, F, Cl, Br, NO₂, phenyl, CO₂R₁₂, or CH₂NHC(═NH)NH₂; or
R₁ and R₂ and the carbons to which they are attached form a benzene ring; or
R₁ and R₂ together are —O—CH₂CH₂—O—, or —O—CH₂—O—; p and q are integers from 1 to 3; R₆, R₇, R₈, R₉, R₁₀, R₁₁ and R₁₂ are radicals that are independently H, (C₁-C₄)-alkyl or phenyl; wherein a is an integer from 1 to 10, b is an integer from 1 to 10, on the condition that |a|−|b|=0. and M comprises a vanadium ion or vanadium ion complex in any possible oxoacid and polyoxometalate form at the appropriate pH.

Thus, it is also part of the invention the treatment and/or prevention of Diabetes mellitus in a mammal, including a human, comprising the administration of any of the above-mentioned compounds of formula (IIA) to a mammal in need of such treatment.

The present invention also relates to a method of treatment and/or prophylaxis of a mammal, including a human, suffering from diabetes mellitus and/or insulin-resistant conditions. This method comprises the administration of a therapeutically effective amount of a vanadium compound of formula (IIA) or a pharmaceutically acceptable solvate thereof, including hydrates, together with pharmaceutically acceptable diluents or carriers, to said patient.

The invention further provides a method of treating an animal (preferably a human) having a disease, disorder or defect characterized by reduced plasma insulin or reduced or absent responsiveness to insulin, comprising administering a therapeutically-effective amount of the compound or pharmaceutical composition of formula IIA to an animal in need of such treatment. In one aspect, the disease, disorder or defect is type I or type II diabetes.

In another embodiment, the invention provides compounds of formula II: 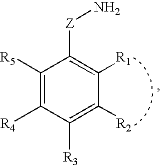
or pharmaceutically acceptable salts thereof, where Z, and R₁-R₅ are as defined for Formula (IIA).

In another aspect, the invention provides methods for preparing compounds of formula (IIA), as well as the intermediates useful in preparing compounds of formula (IIA).

A further aspect of the present invention relates to a pharmaceutical composition comprising a therapeutically effective amount of the compound of formula (IIA) as active ingredient, together with appropriate amounts of pharmaceutical excipients or carriers. The compound of the present invention may be formulated for oral, parenteral, rectal or topical administration.

The invention further comprises kits comprising combinations of the compounds of Formula IIA of the invention, and materials or other reagents useful in preparing or administering pharmaceutical compositions of said compounds. Solutions or diluents provided in the kits of the invention are preferably aqueous solutions or diluents. Most preferably, the kit comprises the compounds of the invention in a single pharmaceutical composition in one or more containers. The container itself may be useful for administering the pharmaceutical compositions of the invention, inter alia, as an inhalant, syringe, pipette, eye dropper or other such apparatus, whereby the pharmaceutical composition of the invention can be administered for example by injection. The pharmaceutical compositions of the invention or components thereof can be provided in dried or lyophilized form, wherein reconstitution is provided by the addition of the appropriate solvent that is advantageously included in the kit. Instructions for preparing or reconstituting the pharmaceutical composition or administration thereof are also advantageously included.

It is advantageous that the arylalkylamine vanadium (V) salts of the present invention are insulin mimickers more powerful than vanadate alone. They are even better than the combinations of vanadium plus amine. Thus, these compounds reduce the toxicity known to result from excess quantities of vanadate. This represents an important advantage of the pharmaceutical compositions containing the arylalkylamine vanadium (V) salts of the present invention in respect of the vanadium compositions known in the art, for the treatment and/or prevention of diabetes mellitus. Furthermore, the fact that the compounds of the present invention are provided as single ingredients is advantageous in respect of the combinations of two ingredients (vanadium plus amine) for the preparation of a pharmaceutical compositions, both from the dosage and the simplicity viewpoints.

Specific preferred embodiments of the present invention will become evident from the following more detailed description of certain preferred embodiments and the claims.

### BRIEF DESCRIPTION OF THE DRAWINGS

FIG. 1 is a graphical illustration of the hexaquis(benzylammonium) decavanadate effects on glucose transport in isolated rat adipocytes. In this Figure, V corresponds to the rate of 2-deoxyglucose transport (expressed relative to the basal rate), and the results are mean±standard error of the mean (SEM). The adipocytes were incubated in the absence of stimulants in the following conditions: basal (1); in the presence of 100 nM insulin (2); in the presence of hexaquis(benzylammonium) decavanadate at concentrations of 0.5 μM (3), 1 μM (4), 2.5 μM (5), 5 μM (6), 10 μM (7), 25 μM (8), 50 μM (9), and 100 μM (10). The cells were also incubated in the presence of the semicarbazide inhibitor (1 mM), and 10 μM hexaquis(benzylammonium) decavanadate (11), 25 μM of hexaquis(benzylammonium) decavanadate (12) or 50 μM hexaquis(benzylammonium) decavanadate (13). In parallel, the cells were incubated in presence of 100 μM of sodium vanadate and 100 μM of benzylamine, in the absence (14) or in the presence of 1 mM of semicarbazide (15).

FIG. 2 is a graphical illustration of the effects of hexaquis(benzylammonium) decavanadate, pentaquis(benzylammonium) decavanadate and tetraquis(benzylammonium) decavanadate on glucose transport in isolated rat adipocytes. V corresponds to the rate of 2-deoxyglucose uptake (expressed as relation with basal group), and the results are mean±standard error mean. The adipocytes were incubated in the absence of stimulants in the following conditions: basal (1); in the presence of 100 nM of insulin (2); in the presence of hexaquis(benzylammonium) decavanadate at concentrations of 10 μM (3) and 25 μM (4), pentaquis(benzylammonium) decavanadate at concentrations of 10 μM (8) and 25 μM (9), and tetraquis(benzylammonium) decavanadate at concentrations of 10 μM (11) and 25 μM (12). The cells were also incubated in the presence of the semicarbazide inhibitor (1 mM) and, 25 μM of hexaquis(benzylammonium) decavanadate (5), 25 μM of pentaquis(benzylammonium) decavanadate (10) or 25 μM of tetraquis(benzylammonium) decavanadate (13). In parallel, the cells were incubated in the presence of 100 μM of sodium vanadate (6) or in the presence of 250 μM of sodium vanadate (7).

FIG. 3 is a graphical illustration of hexaquis(benzylammonium) decavanadate chronic treatment effect on glycemia of diabetic rats by streptozotocin. In FIG. 3, [G] corresponds to the blood concentration of glucose (expressed in mg/dl) measured at different days of treatment (t/d). Diabetic rats were treated, by mini-osmotic pumps, with buffered solution (black diamonds), with hexaquis(benzylammonium) decavanadate (2.5 μmol/kg/day) (black squares) or with identical dose of sodium decavanadate (white circles).

FIG. 4 is a graphical illustration of the chronic and oral treatment with hexaquis(benzylammonium) decavanadate on glycemia of diabetic rats by estreptozotocine. In FIG. 4, [G] corresponds to the blood concentration of glucose (expressed in mg/dl) measured at different days of treatment (t/d). Diabetic rats were treated with a single daily oral dose of hexaquis(benzylammonium) decavanadate (5 μmol/kg/day between day 0 and day 7 marked with an arrow, and 10 μmol/kg/day from 7 days of treatment) (black squares) or with identical dose of sodium decavanadate (black diamonds). Glycemia in non-diabetic rats is also represented in the figure (black triangles).

FIG. 5A through 5C show the stimulatory effects of hexaquis(benzylammonium) decavanadate (B6V10), pentaquis(benzyl ammonium) decavanadate (B5V10) and tetraquis (benzyl ammonium) decavanadate (B4V10) on glucose transport in adipose cells. All values shown are the mean±SEM of 4-5 observations per group, and *, indicates a significant stimulation of 2-DG uptake compared with basal transport value at P<0.001. In FIG. 5A, †, indicates a significant stimulation of 2-DG uptake compared with basal transport value at P<0.05.

FIG. 6A shows chemical structures of advantageous embodiments of the arylalkylamine components of the insulin replacement compounds of the invention.

FIG. 6B shows the effects of vanadium salts of arylalkylamine components of the insulin replacement compounds of the invention on glucose transport by isolated rat adipocytes. *, indicates a significant stimulation of 2-DG uptake in groups incubated in the presence of 25 μM compounds compared with insulin-stimulated transport values at P<0.05.

FIG. 7A through 7E illustrate intracellular signalling pathway activated by hexaquis(benzylammonium) decavanadate in adipose cells and inhibited by phosphatidylinositol 3-kinase inhibitors (FIG. 7E). Values are mean±SEM of 4-5 observations per group. *, indicates a significant stimulation of 2-DG uptake compared with basal transport value at P<0.05.

FIGS. 8A and 8B show the antidiabetic efficacy of administered hexaquis(benzylammonium) decavanadate in rat or mouse models of diabetes. All values are mean±SEM of 6-7 observations. Two way ANOVA indicated the existence of significant differences between the B6V10 and the untreated or V10 groups (in FIG. 8A, P<0.01; FIG. 8B, P<0.001). Bonferroni post-tests for the results shown in FIG. 8A indicated significant differences in the B6V10 group compared to the untreated group from day 8 of treatment, at P<0.01.

FIGS. 9A and 9B illustrate results showing the antidiabetic efficacy of administered hexaquis(benzylammonium) decavanadate in streptozotocin-induced diabetic rat with undetectable circulating insulin. Values are mean±SEM of 6-7 observations. Two way ANOVA indicated the existence of significant differences between the B6V10 and the untreated groups, at P<0.01 (FIG. 9A) or at P<0.05 (FIG. 9B).

### DETAILED DESCRIPTION OF PARTICULAR EMBODIMENTS

In another aspect, the invention provides compounds of formula (I), i.e., compounds of Formula (IIA) having the formula 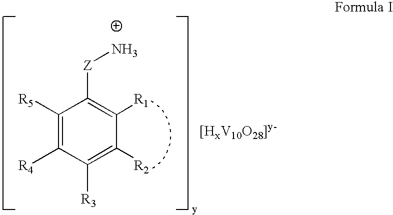
or a pharmaceutically acceptable solvate thereof, including hydrates where Z, R₁-R₅, are as defined for formula (IIA); x is an integer from 0 to 2, and y is an integer from 4 to 6, on the condition that x+y=6.

The invention further provides amines of formula (II) 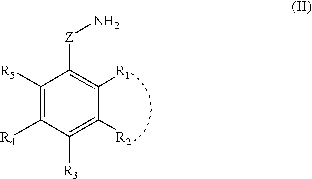
and pharmaceutically acceptable salts thereof, including hydrates where Z, R₁-R₅, are as defined for formula (IIA).

The invention further provides compounds of formula (I), (II) or (IIA), or pharmaceutically acceptable solvates thereof, including hydrates, for the preparation of a medicament for the treatment and/or prevention of diabetes mellitus and/or insulin-resistant conditions in a mammal, such as a human.

In one embodiment, the invention provides compounds of formula (I), (II) or (IIA), where R₁ and R₂ and the carbons to which they are attached form a benzene ring; or R₁ and R₂ are —O—CH₂CH₂—O—, or —O—CH₂—O—.

In a preferred embodiment, the invention provides compounds of formula (I), (II) or (IIA), where R₁, R₂, R₃, R₄, and R₅ are radicals independently selected from H, (C₁-C₆)-alkyl, OH, (C₁-C₆)-alkoxy, O(CH₂)_{q}phenyl, CF₃, OCF₃, F, Cl, Br, and NO₂. In an alternate aspect, R₁ and R₅ are both H.

In another embodiment, the invention provides compounds of formula (I), (II) or (IIA), where R₁, R₂, R₃, R₄ and R₅ are radicals independently selected from H, (C₁-C₄)-alkyl, OH, (C₁-C₄)-alkoxy, O(CH₂)phenyl, CF₃, OCF₃, F, Cl, Br, and NO₂ and Z is —(C₁-C₅ alkyl)- or —(C₁-C₅ alkoxy)-. In an alternate aspect, Z is —(C₁-C₃ alkyl)- or —(C₂-C₄ alkoxy)-. In yet another alternate aspect, Z is —(C₁-C₂ alkyl)- or —(C₂-C₃ alkoxy)-. In still yet another alternate aspect, Z is —(C₁ alkyl)- or —(C₂-alkoxy)-. In yet still another alternate aspect, Z is —CH₂—.

In another aspect, the invention provides compounds of formula (I), (II) or (IIA), where R₃, R₄ and R₅ are radicals independently selected from H, (C₁-C₄)-alkyl, OH, (C₁-C₄)-alkoxy, O(CH₂)phenyl, CF₃, OCF₃, F, Cl, Br, and NO₂ and R₁ and R₂ and the carbons to which they are attached form a benzene ring; and Z is —(C₁-C₅ alkyl)- or —(C₁-C₅ alkoxy)-. In an alternate aspect, Z is —(C₁-C₃ alkyl)- or —(C₂-C₄ alkoxy)-. In yet another alternate aspect, Z is —(C₁-C₂alkyl)- or —(C₂-C₃ alkoxy)-. In still yet another alternate aspect, Z is —(C₁ alkyl)- or —(C₂-alkoxy)-. In yet still another alternate aspect, Z is —CH₂—.

The invention further provides compounds of formula I where 1) when x=0, y=6; 2) when x=1, y=5; and 3) when x=2, y=4.

In one embodiment, the invention provides compounds of formula I or (IIA) that are hexaquis(benzylammonium) decavanadate (V) dihydrate, pentaquis(benzylammonium) decavanadate (V) dihydrate and tetraquis(benzylammonium) decavanadate (V) dihydrate. These embodiments of the decavanadates of the invention are in the oxidation state (V), whether the designation of (V) is included or omitted as set forth herein.

In another aspect, the compounds of formula (I), (II) or (IIA) are insulin replacement compounds. In a preferred aspect, the insulin replacement compounds of formula (IIA) are vanadium salts.

In another aspect, the invention provides compounds of formula (I), (II) or (IIA) where

R₁, R₂, R₃, R₄ and R₅ are independently H, OH, (C₁-C₆)-alkyl, (C₁-C₆)-alkoxy, NR₆R₇, (CH₂)ₚNR₈R₉, O(CH₂)_{q}phenyl, CONR₁₀R₁₁, COR₁₂, NO₂, phenyl, or CO₂R₁₂.

In still another aspect, the invention provides compounds of formula (I), (II) or (IIA) where

R₁, R₂, R₃, R₄ and R₅ are independently H, OH, (C₁-C₆)-alkyl, (C₁-C₆)-alkoxy, NR₆R₇, (CH₂)ₚNR₈R₉, O(CH₂)_{q}phenyl, CONR₁₀R₁₁, COR₁₂, NO₂, phenyl, or CO₂R₁₂;

at least one of R₃, R₄ and R₅ is halogen, CF₃, or OCF₃.

In still yet another aspect, the invention provides compounds of formula (I), (II) or (IIA) where

R₂, R₃, and R₄ are independently H, OH, (C₁-C₆)-alkyl, (C₁-C₆)-alkoxy, NR₆R₇, (CH₂)NR₈R₉, O(CH₂)phenyl, CONR₁₀R₁₁, COR₁₂, NO₂, phenyl, or CO₂R₁₂;

at least one of R₃, R₄ and R₅ is halogen, CF₃, or OCF₃; and

R₁ and R₅ are both H.

In yet still another aspect, the invention provides compounds of formula (I), (II) or (IIA) where

R₃, and R₄ are independently H, OH, (C₁-C₆)-alkyl, (C₁-C₆)-alkoxy, NR₆R₇, (CH₂)ₚNR₈R₉, O(CH₂)_{q}phenyl, CONR₁₀R₁₁, COR₁₂, NO₂, phenyl, or CO₂R₁₂;

R₅ is H;

and R₁ and R₂ are bound together forming a ring with a fused benzene; or

R₁ and R₂ are —O—CH₂CH₂—O—, or —O—CH₂—O—. p and q are integers from 1 to 2; R₆, R₇, R₈, R₉, R₁₀, R₁₁ and R₁₂ are radicals that are independently H, or (C₁-C₄)-alkyl; Z is —(C₁-C₆ alkyl)- or —(C₁-C₆ alkoxy)-. In a further aspect, Z is —(C₁-C₅ alkyl)- or —(C₁-C₅ alkoxy)-. In an alternate aspect, Z is —(C₁-C₃ alkyl)- or —(C₂-C₄ alkoxy)-. In yet another alternate aspect, Z is —(C₁-C₂ alkyl)- or —(C₂-C₃ alkoxy)-. In still yet another alternate aspect, Z is —(C₁ alkyl)- or —(C₂-alkoxy)-. In yet still another alternate aspect, Z is —CH₂—. In compounds of formula (I), a is an integer from 1 to 10, and b is an integer from 1 to 10, on the condition that |a|−|b|=0; and M comprises a vanadium ion or vanadium ion complex in any possible oxoacid and polyoxometalate form at the appropriate pH.

In another aspect, the invention provides compounds where R₁, R₂, R₃, R₄ and R₅ are independently selected from H, OH, (C₁-C₆)-alkyl, (C₁-C₆)-alkoxy, NR₆R₇, (CH₂)ₚNR₈R₉, O(CH₂)_{q}phenyl, CONR₁₀R₁₁, COR₁₂, CF₃, OCF₃, F, Cl, Br, NO₂, phenyl, CO₂R₁₂, and CH₂NHC(═NH)NH₂; or

R₁ and R₂ and the carbons to which they are attached form a benzene ring; or

R₁ and R₂ are —O—CH₂CH₂—O—, or —O—CH₂—O—; p and q are integers from 1 to 3;

R₆, R₇, R₈, R₉, R₁₀, R₁₁ and R₁₂ are radicals that are independently H, (C₁-C₄)-alkyl or phenyl; and Z is —(C₁-C₆ alkoxy)- or —(C₁-C₆ alkyl)-. The invention further provides compounds where Z is —(C₁-C₅ alkyl)- or —(C₁-C₅ alkoxy)-. In an alternate aspect, Z is —(C₁-C₃ alkyl)- or —(C₂-C₄ alkoxy)-. In yet another alternate aspect, Z is —(C₁-C₂ alkyl)- or —(C₂-C₃ alkoxy)-. In still yet another alternate aspect, Z is —(C₁ alkyl)- or —(C₂-alkoxy)-. In yet still another alternate aspect, Z is —CH₂—.

In one preferred embodiment, the invention provides compounds of formula (I), (II), or (IIA) where R₁, R₂, R₃, R₄ and R₅ are radicals that are independently H, OH, (C₁-C₃)-alkyl, (C₁-C₃)-alkoxy, NR₆R₇, (CH₂)ₚNR₈R₉, O(CH₂)_{q}Phenyl, CONR₁₀R₁₁, COR₁₂, NO₂, or CH₂NHC(═NH)NH₂;

at least one of R₃, R₄ and R₅ are halogen, CF₃, or OCF₃;

or alternatively R₁ and R₂ are bound together forming a ring with a fused benzene; p and q are integers from 1 to 3; R₆, R₇, R₈, R₉, R₁₀, R₁₁ and R₁₂ are independently H, or (C₁-C₄)-alkyl; and Z is —(C₁-C₆ alkoxy)- or —(C₁-C₆ alkyl)-. The invention further provides compounds where Z is —(C₁-C₅ alkyl)- or —(C₁-C₅ alkoxy)-. In an alternate aspect, Z is —(C₁-C₃ alkyl)- or —(C₂-C₄ alkoxy)-. In yet another alternate aspect, Z is —(C₁-C₂ alkyl)- or —(C₂-C₃ alkoxy)-. In still yet another alternate aspect, Z is —(C₁ alkyl)- or —(C₂-alkoxy)-. In yet still another alternate aspect, Z is —CH₂—.

In still another aspect, the invention provides compounds of formula (I), (II) or (IIA), where R₁, R₂, R₃, R₄ and R₅ are radicals independently selected from H, OH, (C₁-C₆)-alkyl, (C₁-C₆)-alkoxy, O(CH₂)_{q}Phenyl, COR₁₂, CF₃, OCF₃, F, Cl, Br, NO₂, phenyl, and CO₂R₁₂.

In yet another aspect, the invention provides compounds of formula (I), (II) or (IIA), where R₁ and R₂ are —O—CH₂CH₂—O—, or —O—CH₂—O—.

In an alternative aspect, the invention provides compounds of formula (I), (II) or (IIA) where Z is —OCH₂CH₂— or —OCH₂CH₂CH₂—.

In another preferred embodiment, the invention provides compounds of formula (I), (II) or (IIA) where R₄ is H and R₃ and R₅ are independently H, OH, OMe, F, Cl, Br, NO₂, CF₃ or OCF₃. In a more preferred embodiment, Z is —CH₂—.

In an alternatively preferred embodiment, the invention provides compounds of formula (I), (II) or (IIA) where Z is —(C₂-C₄ alkyl)-; and at least one of R₁, R₂, R₃, R₄ and R₅ is F, CF₃ or OCF₃. Alternatively, R₅ is H and R₄ is OMe, F, Cl, Br, CF₃, OCF₃, or C₁-C₂ alkyl.

In yet another aspect, the invention provides for compounds of formula (I), or (IIA), wherein, Z is —(C₂-C₄ alkyl)-; M is V₁₀O₂₈, and a is 6 and b is 6. More preferably, R₄ is H and R₃ and R₅ are independently H, OH, OMe, F, Cl, Br, NO₂, CF₃ or OCF₃. Even more preferably Z is —C₃-C₄ alkyl-, and R₃ and R₅ are independently H, F, CF₃, or OCF₃.

In one aspect, the halogen substituent is fluorine, and the fluorine derivatives such as OCF₃ and CF₃ and the alkylamine substituent are is positions ortho or para to one another on the benzene ring. In alternative embodiments, the benzene ring can be substituted at any other position (other than the halogen and alkylamine substituents) with a group including but not limited to alkyl (including cycloalkyl and heterocycloalkyl) and substituted alkyl (substituted with 1 or 2 groups that are independently C₁-C₄ alkoxy, halogen, OH or CO₂H) and C₃-C₈ cycloalkyl; C₂-C₆ acyl and substituted acyl (substituted with 1 or 2 groups that are independently C₁-C₄ alkoxy, halogen, OH or CO₂H); aryl (such as phenyl or naphthyl, with phenyl being preferred), heteroaryl (such as pyrrolyl or pyridyl) and substituted aryl (substituted with 1 or groups that are independently C₁-C₄ alkoxy, halogen, OH or CO₂H); halogen; and nitro-, hydroxyl, sulfo- and sulfonyl-groups. In additional alternative embodiments, the benzene ring can be substituted with or fused to cyclopentyl and cycloheptyl rings, or heteroatom-substituted embodiments thereof, including, but not limited to pyrrole, furan, thiophene, imidazole, pyrazole, thiazole, and oxazole, and substituted derivatives thereof (substituted with 1 or 2 groups that are C₁-C₄ alkoxy, halogen, OH or CO₂H), as well as naphthyl, substituted naphthyl (substituted with 1 or 2 groups that are C₁-C₄ alkoxy, halogen, OH or CO₂H), and heteroatom-substituted naphthyl groups (where N is the preferred heteroatom), or phenols.

The invention further provides pharmaceutical compositions comprising the halogen-substituted arylalkylamines of Formula (I), (II) or (IIA), and more preferably the vanadium salt thereof, formulated with pharmaceutically-acceptable diluents, solvents, excipients or adjuvants, or combinations thereof.

This invention further provides compounds and pharmaceutical compositions thereof that are antidiabetic, insulin sensitizer, insulin mimetic or insulin replacement compounds.

This invention provides compounds and salts thereof, as well as pharmaceutical compositions thereof for treating diabetes, preferably human diabetes type I and/or type II and in particular insulin-resistant diabetes. The compounds, salts and pharmaceutical compositions provided by the invention are antidiabetic, insulin sensitizer, insulin mimetic or insulin replacement compounds, and are preferably provided as salts of arylalkylamines, more preferably provided as salts of halogenated arylalkylamines, and most preferably vanadium salts thereof or mixtures of said arylalkylamines and vanadium, selenium, molybdenum or tungsten salts. Methods for formulating the pharmaceutical compositions of the compounds of the invention and methods for administering said compounds or pharmaceutical compositions of the invention are also provided.

The invention specifically provides arylalkylamines and vanadium salts thereof as non-peptide small molecules that exert potent insulin mimetic effects in adipocytes in vitro and insulin replacement effects in vivo. Without being bound to any theory or mechanism of action of these compounds, at least certain of these compounds appear to act in the insulin signalling cascade at a point downstream from the insulin receptor, by triggering insulin signalling characterized by rapid activation of Akt in the absence of insulin receptor phosphorylation. A particular compound provided by the invention, hexaquis(benzyl ammonium) decavanadate ((C₇H₁₀N)₆V₁₀O₂₈.2H₂O; B6V10) caused amelioration of glucose tolerance and improved plasma insulin levels when administered to insulin high-fat fed mice. Chronic oral or subcutaneous administration of B6V10 to rat or mouse models of diabetes resulted in significant lowering in blood glucose levels. Notably, B6V10 showed antidiabetic effects in severely diabetic rats with undetectable circulating insulin. In addition, B6V10 inhibited adipocyte lipolysis and normalized the plasma concentrations of free fatty acids, triglycerides and cholesterol of diabetic rats. These results demonstrated that the compounds provided by the invention are insulin replacement compounds that activate insulin signalling downstream of the insulin receptor. Such compounds are of therapeutic use in diabetes, severe insulin resistance or dyslipidemia, where disease, defect or disorder is associated with or caused by inhibition or abolition of insulin receptor responsiveness.

In still yet another aspect, the invention provides compounds of formula (IIA), where the amine of formula (II) that is complexed to the vanadium is (4-fluorophenyl)methanamine, p-tolylmethanamine, (4-ethylphenyl)methanamine, (4-tert-butylphenyl)methanamine, (4-isopropylphenyl)methanamine, (4-(trifluoromethyl)phenyl)methanamine, (4-(trifluoromethoxy)phenyl)methanamine, (2,4-dimethoxyphenyl)methanamine, (4-butylphenyl)methanamine, 4-(aminomethyl)-2-bromophenol, 2-(aminomethyl)-6-nitrophenol, biphenyl-4-ylmethanamine, (3-fluoro-4-methylphenyl)methanamine, (3-fluoro-5-methoxyphenyl)methanamine, 2-(aminomethyl)-3-methoxyphenol, (4-(benzyloxy)phenyl)methanamine, (2,3-dihydrobenzo[b][1,4]dioxin-5-yl)methanamine, (4-methoxyphenyl)methanamine, (2-fluorophenyl)methanamine, (3,5-dimethoxyphenyl)methanamine, 1,4-phenylenedimethanamine, 1,3-phenylenedimethanamine, 4-(aminomethyl)benzoic acid, (4-methoxynaphthalen-1-yl)methanamine, 1-(aminomethyl)naphthalen-2-ol, 1-(naphthalen-1-yl)ethanamine, 2-phenylethanamine, 3-phenylpropan-1-amine, 2-phenoxyethanamine, 4-phenylbutan-1-amine, 2-(4-fluorophenyl)ethanamine, 4-(2-aminoethyl)benzene-1,2-diol or 2-(3,4-dimethoxyphenyl)ethanamine.

In an alternative aspect of the invention, the vanadium salt is complexed with (4-methoxy-1H-indol-3-yl)methanamine.

In yet another aspect, the pharmaceutical composition comprises the vanadium salt of (4-methoxy-1H-indol-3-yl)methanamine.

In still another aspect, the invention provides methods of treating diabetes comprising administering a therapeutically effective amount of the vanadium salt of (4-methoxy-1H-indol-3-yl)methanamine or a pharmaceutically acceptable salt thereof, to a patient in need of such treatment. In still another aspect, the invention provides compounds of formula (IIA), where the arylalkylamines is p-tolylmethanamine, (4-ethylphenyl)methanamine, (4-tert-butylphenyl)methanamine, (4-isopropylphenyl)methanamine, (4-butylphenyl)methanamine, biphenyl-4-ylmethanamine, (4-(benzyloxy)phenyl)methanamine, 2-phenylethanamine, 3-phenylpropan-1-amine, 2-phenoxyethanamine, or 4-phenylbutan-1-amine.

In yet still another aspect, the invention provides compounds of formula (IIA), where the arylalkylamines is (4-fluorophenyl)methanamine, (4-(trifluoromethyl)phenyl)methanamine, (4-(trifluoromethoxy)phenyl)methanamine, (3-fluoro-4-methylphenyl)methanamine, (3-fluoro-5-methoxyphenyl)methanamine, (2-fluorophenyl)methanamine, or 2-(4-fluorophenyl)ethanamine.

In still yet another aspect, the invention provides compounds of formula (IIA), where the arylalkylamines is (2,4-dimethoxyphenyl)methanamine, 4-(aminomethyl)-2-bromophenol, 2-(aminomethyl)-6-nitrophenol, 2-(aminomethyl)-3-methoxyphenol, (2,3-dihydrobenzo[b][1,4]dioxin-5-yl)methanamine, (3,5-dimethoxyphenyl)methanamine, 1,4-phenylenedimethanamine, 1,3-phenylenedimethanamine, 4-(aminomethyl)benzoic acid, 4-(2-aminoethyl)benzene-1,2-diol, 2-(3,4-dimethoxyphenyl)ethanamine.

In yet another aspect, the invention provides compounds of formula (IIA), where the arylalkylamines is biphenyl-4-ylmethanamine, (2,3-dihydrobenzo[b][1,4]dioxin-5-yl)methanamine, 4-(aminomethyl)benzoic acid, or 2-phenoxyethanamine.

In another aspect, the invention provides methods of making compounds of formula IIA, 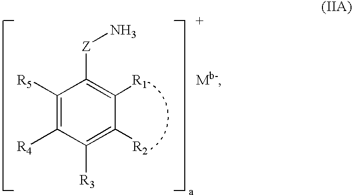
where M, a, b, Z, and R1-R5 are as defined for formula (IIA), the method comprising
a) reacting an amine of formula (II) 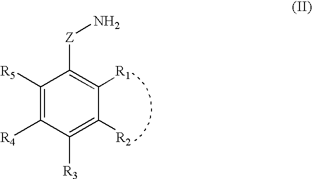
where Z and R₁-R₅ are as defined for formula (IIA), with an alkaline metal vanadate in an inert solvent at a appropriate acidity, and
b) recovering the compound of formula (IIA) from the reaction media.

Preferably, the alkaline metal vanadate is sodium vanadate. The reaction can be conveniently carried out in water, or in mixtures of water with an organic solvent selected from (C₁-C₅)-aliphatic monoalcohols such as methanol, ethanol and 1-butanol, and (C₃-C₇)-aliphatic ketones such as acetone, 2-propanone and 4-methyl-2-pentanone. Preferably, the acidity corresponds to an effective pH value of the reaction between 2 and 7.5.

In particular embodiments, the invention provides vanadate salts of arylalkylamines of formula (II) as antidiabetic agents and/or insulin sensitizers, which can be administered as a single ingredient, and which are even better insulin mimickers than the combinations of vanadium plus amine.

The invention further provides compounds or a pharmaceutically acceptable solvate thereof, including hydrates, of formulas (I), (II), or (IIA) for the preparation of a medicament for the treatment and/or prevention of diabetes mellitus and/or insulin-resistant conditions in a mammal, including a human. Thus, it is also part of the invention the treatment and/or prevention of diabetes mellitus in a mammal, including a human, comprising the administration of any of the above-mentioned compounds of formula (IIA), (II), or (I).

The invention further provides a method of treating an animal having a disease, disorder or defect characterized by reduced plasma insulin or reduced or absent responsiveness to insulin by administering a therapeutically-effective amount of the compound or pharmaceutical composition of formula (I), (II), or (IIA). In a preferred embodiment, the animal is a human. In a further preferred embodiment, the disorder, disease or defect is diabetes.

In another embodiment, the method of treatment comprises administering the free arylalkylamine of formula II to a patient in need of such treatment. Preferably, the method of treatment comprises the administration of a compound of formula IIA or formula I. Alternatively the method of treatment comprises a compound comprising (4-methoxy-1H-indol-3-yl)methanamine complexed to a vanadium salt. Most preferably, the arylalkylamine is 4-phenylbutan-1-amine, (4-fluorophenyl)-methanamine, or (3-fluoro-4-methylphenyl)methanamine.

Alternatively, the invention provides the vanadium salt of 4-phenylbutan-1-amine, (4-fluorophenyl)-methanamine, or (3-fluoro-4-methylphenyl)methanamine.

The invention also provides for pharmaceutical compositions comprising therapeutically effective amount of the compound or salt of formula (I), (II), or (IIA).

In a preferred embodiment, the composition comprises the compound of formula IIA.

In yet another preferred embodiment, the pharmaceutical composition comprises the compound of formula I.

By “alkyl”, “lower alkyl”, and “C₁-C₆ alkyl” in the present invention is meant straight or branched chain alkyl groups having 1-6 carbon atoms, such as, methyl, ethyl, propyl, isopropyl, n-butyl, sec-butyl, tert-butyl, pentyl, 2-pentyl, isopentyl, neopentyl, hexyl, 2-hexyl, 3-hexyl, and 3-methylpentyl.

By “alkoxy”, “lower alkoxy”, and “C₁-C₆ alkoxy” in the present invention is meant straight or branched chain alkoxy groups having 1-6 carbon atoms, such as, for example, methoxy, ethoxy, propoxy, isopropoxy, n-butoxy, sec-butoxy, tert-butoxy, pentoxy, 2-pentyl, isopentoxy, neopentoxy, hexoxy, 2-hexoxy, 3-hexoxy, and 3-methylpentoxy.

By the term “halogen” in the present invention is meant fluorine, bromine, chlorine, and iodine.

By “cycloalkyl”, e.g., C₃-C₇ cycloalkyl, in the present invention is meant cycloalkyl groups having 3-7 atoms such as, for example cyclopropyl, cyclobutyl, cyclopentyl, cyclohexyl, and cycloheptyl.

By “aryl” is meant an aromatic carbocyclic group having a single ring (e.g., phenyl), multiple rings (e.g., biphenyl), or multiple condensed rings in which at least one is aromatic, (e.g., 1,2,3,4-tetrahydronaphthyl, naphthyl, anthryl, or phenanthryl), which is optionally mono-, di-, or trisubstituted with, e.g., halogen, C₁-C₆ alkyl, C₁-C₆ alkoxy, C₁-C₆ alkylthio, trifluoromethyl, C₁-C₆ acyloxy, aryl, heteroaryl, and hydroxy. Preferred aryl groups include phenyl, indanyl, biphenyl, and naphthyl, each of which is optionally substituted as defined herein. More preferred aryl groups include phenyl and naphthyl, each of which is optionally substituted as defined herein.

By “heteroaryl” is meant an aromatic ring or aromatic ring system, wherein each ring contains of 5-, 6-, or 7-members wherein at least one and up to four ring members are selected from nitrogen, oxygen, or sulfur. Such heteroaryl groups include, for example, thienyl, furanyl, thiazolyl, imidazolyl, (is)oxazolyl, pyridyl, pyrimidinyl, (iso)quinolinyl, indolyl, napthyridinyl, benzimidazolyl, and benzoxazolyl. Preferred heteroaryls are thiazolyl, pyrimidinyl, pyrimidin-2-yl, indolyl, pyridyl, 1-imidazolyl, 2-thienyl, 1-, or 2-quinolinyl, 1-, or 2-isoquinolinyl, 1-, or 2-tetrahydro isoquinolinyl, 2- or 3-furanyl and 2-tetrahydrofuranyl.

By “heterocycloalkyl,” is meant one or more carbocyclic ring systems of 3, 4, 5, 6, or 7-membered rings which includes fused ring systems of 9-11 atoms containing at least one and up to four heteroatoms independently selected from nitrogen, oxygen, and sulfur. Preferred heterocycles of the present invention include morpholinyl, thiomorpholinyl, thiomorpholinyl S-oxide, thiomorpholinyl S,S-dioxide, piperazinyl, homopiperazinyl, pyrrolidinyl, pyrrolinyl, tetrahydropyranyl, piperidinyl, tetrahydrofuranyl, tetrahydrothienyl, homopiperidinyl, homomorpholinyl, tetrahydroquinolynyl, homothiomorpholinyl, homothiomorpholinyl S,S-dioxide, oxazolidinonyl, dihydropyrazolyl, dihydropyrrolyl, dihydropyrazinyl, dihydropyridinyl, dihydropyrimidinyl, dihydrofuryl, dihydropyranyl, azepanyl, diazepanyl, tetrahydrothienyl S-oxide, tetrahydrothienyl S,S-dioxide and homothiomorpholinyl S-oxide.

The amines of formula (II) can be prepared by several methods. A first general method for the preparation of the amines of formula (I) is summarized in Scheme I. It comprises reacting an aldehyde of formula (III) with hydroxylamine, where R₁, R₂, R₃, R₄, and R₅ have the above-mentioned meaning, followed by reducing the obtained oxime. The reaction of the aldehyde with the hydroxylamine is carried out in a suitable reaction-inert solvent, preferably at the reflux temperature of the reaction mixture. The reduction of the oxime is preferably carried out by catalytic hydrogenation using PtO₂ as catalyst in an inert solvent.

A further general method for the preparation of the amines of formula (II) is summarized in Scheme II. It comprises carrying out a reductive amination by solid phase synthesis of an aldehyde of formula (III) with an amino group anchored onto a polymeric support. The method include the following steps: a) removing the protecting group (e.g. 9-fluorenylmethoxycarbonyl) of the polymeric support which contains an acid-labile linker, with reactants commonly used in peptides synthesis; b) condensing the aldehyde with the free amino function of the polymeric support using a dehydrating agent; c) reducing the imine with a reducing agent such as lithium borohydride; and, d) releasing the compound of the polymeric support in acidic conditions (e.g. trifluoroacetic acid).

Polystyrene and polyethylenglycol grafted to polystyrene are among the compounds which can be used as polymeric supports. These supports include an acid-labile linker such as XAL(((9-(amino)xanthen-2-yl)oxy)butanoic acid handle), and Rink (p-((R,S)-α-(1-(9H-fluoren-9-yl)-methoxyformamido)-2,4-dimethoxybenzyl)-phenoxyacetic acid).

R₁, R₂, R₃, R₄, and R₅ are substituents selected from the group defined above or intermediate forms thereof which can be transformed into such substituents. The transformation is carried out after reducing the imine, being the amino group protected with a protecting group such as 2-nitrophenylsulphenyl. For example an hydroxyl substituent can be transformed into an alkoxy substituent by alkylation with an alkylating agent in the presence of a base.

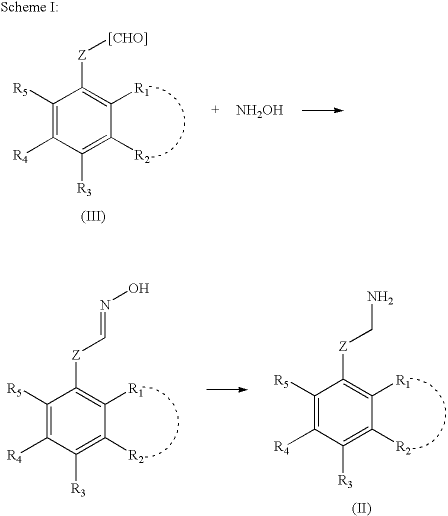

In scheme I, the variables Z and R₁-R₅ are as defined as in formula (II). Scheme 1 demonstrates one possible method for preparing the amines of the invention. Compound (III) is treated with hydroxylamine in a solvent and the resulting hydroxylamine is reduced to form the desired amine.

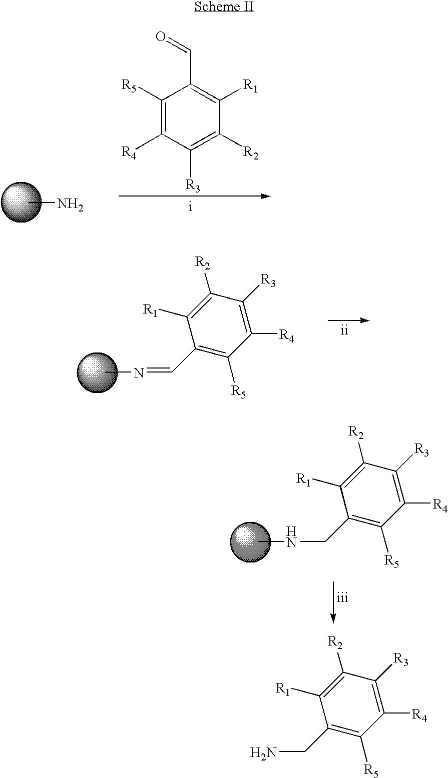

^{a}Reagents and conditions: (i) 5 equiv RCHO/TMOF 2 h at rt, (ii) 5 equiv LiBH₄/THFanhydrous 5 h at 65° C., (ii) TFA

In scheme II, the variable Z is —CH₂—, and R₁-R₅ are as defined as in formula (I).

The synthetic approach started from the condensation of the corresponding aldehyde to the resin in TMOF as a condensing agent and solvent, followed by the reduction of the aldimine with LiBH₃CN in dry THF. The subsequent cleavage gave the final arylkalkylamines, which were analyzed by HPLC/MS (scheme 1).

General method for the preparation of arylalkylamine derivatives by reductive amination on Rink resins. Each aldehyde (15 eq.) was condensed with Rink resin (100 mg, 0.7 mmol/g) in TMOF (1 mL) and the mixture was stirred under Ar for 2 h at 25° C. to give the corresponding aldimine. The resin was filtered off and washed with TMOF (5×1 min) and then anhydrous THF (6×1 min). Next, LiBH₄ (15 eq., 40 μl) in dry THF was added under Ar atmosphere and the mixture was stirred for 5 h at 65° C. to yield the corresponding secondary amine-bound resins. The resins were filtered and washed several times with THF (10×1 min), H₂O (10×1 min), MeOH (10×1 min) and DCM (10×1 min). The amine was cleaved from the resin with TFA-DCM (95:5) at 25° C. The solution was filtered off and evaporated to dryness under low pressure. The corresponding arylalkylamines were analyzed by HPLC-MS, which showed that the purity attained was over 70% in all cases.

N-(3-Aminomethyl-benzyl)-carbamate resin. Wang resin (1 g, 1.1 mmol/gr) was treated with 4-nitrophenyl chloroformate (5 eq., 0.97 g) and DIEA (5 eq., 57 μL) in NMP (10 mL) overnight at 60° C. The resin was then washed with NMP (5×1), DMF (5×1) and DCM (5×1). The extension of the reaction was followed by the hydroxyl test and IR. The corresponding 4-nitrophenylcarbonate resin was reacted with 1,3-bis(aminomethyl)benzene (5 eq., 59 μL) and DIEA (15 eq., 2.32 mL) in DCM in 10 mL DCM overnight at room temperature. The resin was then washed with DMF (5×1) and DCM (5×1) to eliminate excess amine. The extension of the reaction was followed by IR and Kaiser (ninhydrin test) test.

General method for the synthesis of N-(3-aminomethyl-benzyl)-sulfonamide derivatives. N-(3-Aminomethyl-benzyl)-carbamate resin (100 mg, 1.1 mmol/gr) was reacted with the corresponding sulfonide chloride derivative (5 eq.) and DIEA (5 eq.) in DCM overnight. The resin was filtered and washed with DCM (5×1 min), and the course of the reaction was evaluated by the ninhydrin test. The N-(3-aminomethyl-benzyl)-sulfonamide derivative resin was cleaved from the resin with TFA-DCM (95:5) for 2 h at room temperature (rt). The solution was filtered off and evaporated to dryness under low pressure. The corresponding N-(3-aminomethyl-benzyl)-sulfonamide derivative was analyzed by HPLC-MS, which showed that the purity attained was over 80% in all cases.

General method for the synthesis of N-(3-aminomethyl-benzyl)-acylamide derivatives. N-(3-Aminomethyl-benzyl)-carbamate resin (100 mg, 1.1 mmol/gr) was reacted for 2 h with RCOOH/HOBt/DIPCDI (3 eq.:3 eq.:3 eq.) as acylating mixture in DMF for 2 h at rt. The resin was filtered and washed with DMF (5×1 min) and DCM (5×1 min), and the course of the reaction was followed by the Kaiser test. The N-(3-aminomethyl-benzyl)-acetamide derivative carbamate-resin was cleaved with TFA-DCM (95:5) for 2 h at rt. The solution was filtered off and evaporated under low pressure to dryness. The corresponding N-(3-aminomethyl-benzyl)-acylamide derivatives were analyzed by HPLC-MS, which showed that the purity attained was over 75% in all cases.

The antidiabetic, insulin sensitizer, insulin mimetic or insulin replacement compounds of the invention are provided for simultaneous, separate or sequential administration, and comprise an arylalkylamine of Formula I and a salt, most preferably a vanadium salt, preferably in admixture with pharmaceutically acceptable excipients or carriers.

Salts of the arylalkylamines provided by the invention are preferably vanadium salts and are preferably chemical compounds comprising, as a part of its structure, vanadium. Although said salts may occur in a charged or a non-charged form, the salts provided by the invention typically comprise a salt of the metal ion. If the salt is positively charged, the salt my further comprise a counter ion, which is then negatively charged, e.g. F⁻, Cl⁻, Br⁻, I⁻, OH—, or any pharmaceutically acceptable organic or inorganic ionic species which carries a negative charge. If the salt is negatively charged, the salt may further comprise a counter ion, which is positively charged. Positively charged counter ions typically comprise metals from alkali- or earth alkali metals, such as sodium, potassium, magnesium, calcium, as well as other positively charged ions such as ammonium or any pharmaceutically acceptable organic or inorganic ionic species which carries a positive charge. If the positively or negatively charged salt is combined with a negatively or positively charged counter ion, preferably a neutral salt is obtained.

Vanadium (V) is typically present in the inventive compounds and pharmaceutical compositions thereof in its oxidation state (+4) or (+5), preferably with a tetrahedric or octahedral coordination sphere. In the inventive composition, vandium, preferably V⁴⁺ or V⁵⁺, can occur along with any chemical entity. The cations V⁴⁺ or V⁵⁺ preferably occur as vanadium compounds selected from vanadates (IV)/(V), pervanadates, polyoxometalates, vanadyl salts and/or vanadyl complexes hydrated or not. The cation V⁴⁺ or V⁵⁺ is always accompanied with a chemical moiety partially formed by a coordination sphere around the atom of V(IV/V). The coordination sphere can be formed by inorganic ligands (oxide, hydroxide, peroxide, etc) as, for example, in the case of the vanadate anion (coordination sphere formed by four oxide ions), or in the case of peroxytungstates (coordination sphere formed by mixtures of oxide and peroxide ions). The coordination sphere can also be formed by organic ligands which are molecules or ions attached to V(IV/V) atom through O, S or N atoms belonging to different pharmaceutically acceptable organic compounds and coordination sphere (e.g. pharmaceutically acceptable alcohols, thiols, carboxylic acids, amines, amino acids, or N-containing hereocycles). Mixed inorganic/organic coordination spheres are also possible. When the structure formed by the V(IV/V) atom and its coordination sphere is not neutral, the term “chemical moiety” also includes any pharmaceutically acceptable ionic species which renders the entire V(IV/V)compound neutral. For example, the vanadate anion is always accompanied by a cation (e.g. ammonium, sodium, potassium, magnesium, or calcium or arylalkylamine) to form a neutral vanadate salts. In aqueous solutions, the oxovanadates presented at different pH in reliably detectable proportions are [VO₄]³⁻, [HVO₄]²⁻[H₂VO₄]³⁻, [V₂O₇]⁴⁻, [HV₂O₇]³⁻, [V₃O₉]³⁻, [V₄O₁₂]⁴⁻, the decavanadate [V₁₀O₂₈]⁶⁻ nude or in various states of protonation, and [VO₂]⁺. In the case where the oxanion appears as a neutral form (eg. M^{II}VO₄) M^{II} is a metal selected from earth alkali metals, e.g. Mg²⁺ or Ca²⁺. Hydrates of vanadate (VI) compounds are common (e.g. the hydrate of sodium vanadate), and their use is also considered to be within of this invention.

Any of the afore mentioned compounds may be present in the inventive compounds and pharmaceutical compositions thereof in a soluble and or solubilized form. Therefore, the vanadium (V)/VI) compounds and their (cationic) moieties as contained in the inventive compounds and pharmaceutical compositions thereof are preferably selected such as to obtain a soluble compound.

Furthermore, said vanadium compounds are preferably pharmaceutically acceptable compounds. In the context of this invention, the term “a pharmaceutically acceptable compound” is intended to include any of the aforementioned compounds attached to a chemical structure that is pharmaceutically acceptable by itself. The term “pharmaceutically acceptable compound” also included any pharmaceutically acceptable solvate (e.g. hydrate) of the compounds as contained in the inventive compounds.

As provided herein, the compounds and pharmaceutical compositions of the invention comprise an arylalkylamine, or in one preferred embodiment, a halogenated derivative of an arylalkylamine, such as tyramine, benzylamine, 2-(4-fluoro-phenyl)-ethylamine, 4-fluoro-benzylamine, 3-phenyl-propylamine, 4-phenyl-butylamine, 2,3-dimethoxybenzylamine, 1-naphtalenemethylamine, deoxyepinephrine, epinephrine, norepinephrine, dopamine, histamine, β-phenlethylamine, N-acetylputrescine, tryptamine, n-octylamine, n-pentylamine, kynuramine, 3-methoxytyramine, and n-decylamine.

Provided herein are exemplary arylalkylamines prepared from benzylamine and vanadate: hexaquis(benzylammonium) decavanadate ((C₇H₁₀N)₆V₁₀O₂₈.2H₂O; termed “B6V10” herein), pentaquis(benzylammonium) decavanadate ((C₇H₁₀N)₅HV₁₀O₂₈; “B5V10”) and tetraquis(benzylammonium) decavanadate ((C₇H₁₀N)₄H₂V₁₀O₂₈; “B4V10”), which were extensively characterized in solution and in the solid state by IR spectrum, ⁵¹V-NMR, ¹H/¹³C-NMR, elemental analysis and X-Ray diffraction analysis. The profile of biological effects of arylalkylamine and salts thereof demonstrated that these small non-peptide molecules are capable of replacing the in vivo function of insulin. Without being bound by any particular theory or mechanism, arylalkylamine vanadium salts activate the intracellular insulin signalling pathway downstream of the insulin receptor. Thus, the intracellular activation followed by these compounds was characterized by rapid phosphorylation of protein kinase B in both Thr³⁰⁸ and Ser⁴⁷³ that occurred in the absence of activation of insulin receptors. Based on these observations, arylalkylamine vanadium salts can trigger a number of signalling events in adipose cells, which include initial inhibition of protein tyrosine phosphatase activity, followed by activation of phosphatidylinositol 3-kinase and protein kinase B/Akt, that occurs in the absence of activation of insulin receptor tyrosine kinase. As a consequence, the compounds of the invention are insulin replacement agents which can act in individuals highly resistant to insulin or that otherwise suffer from disease, disorder or defect related to or associated with inhibition or abolition of insulin receptor responsiveness.

Consequently, arylalkylamine vanadium salts of the invention have a profound activity in different tissues of relevance for metabolic homeostasis. Arylalkylamine vanadium salts of the invention activate glucose transport and inhibit lipolysis in adipose cells. Skeletal muscle also responds to arylalkylamine vanadium salts by acutely enhancing glucose uptake and by increasing insulin responsiveness in animal models of diabetes associated to insulin resistance (Abella et al., 2003, Diabetes 52: 1004-1013). The components of arylalkylamine vanadium salts also enhance insulin secretion in pancreatic islets obtained from Goto-Kakizaki diabetic rats, indicating the capacity of arylalkylamine vanadium salts of the invention to enhance insulin secretion by β-pancreatic cells (Abella et al., 2003, Id). In support of this aspect of the compounds and pharmaceutical compositions of this invention, acute administration of hexaquis(benzylammonium) decavanadate (B6V10) ameliorated the pattern of plasma insulin levels in mice made glucose intolerant by a high fat diet.

In vivo chronic oral or subcutaneous treatment with B6V10 ameliorated hyperglycemia in rats made diabetic by streptozotocin administration (45 mg/kg). Similarly, chronic B6V10 treatment ameliorated glycemia in obese diabetic db/db mice. Even more notable, B6V10 was markedly effective in lowering hyperglycemia in diabetic rats with undetectable circulating insulin after administration of a very large dose of streptozotocin (100 mg/kg). B6V10 treatment did not cause hypoglycemia in animals and at the doses used did not show toxicological side effects. In this regard, it must be mentioned that the effective dose of B6V10 as anti-diabetic agent represents a very low dose of vanadium, which is below the “no-observed-adverse-effects level” dose of vanadium in rodents (Trumbo et al., 2001, J. Amer. Dietetic Assoc. 101: 294-301 (2001). Based on this evidence, arylalkylamine vanadium salts represent a novel class of antidiabetic agents of therapeutic value in the treatment of type 1 diabetes, type 2 diabetes and a subset of type 2 diabetic patients characterized by severe insulin resistance. Because arylalkylamine vanadium salts appear to stimulate insulin signalling at a step downstream of the insulin receptor, these compounds are appropriate for the treatment of conditions characterized by the lack of insulin receptor activity such as in patients affected by type A or type B insulin resistance syndromes or in other types of severe insulin resistance which are refractory to treatment with insulin sensitizers and in which the only effective therapy is large doses of insulin (Vestergaard et al., 2001, J. Intern. Med. 250: 406-414).

For the present invention it is advantageous that the effective concentrations of the inventive compounds and pharmaceutical compositions thereof comprising arylalkylamines and pharmaceutically acceptable salts thereof and to their use as antidiabetic, insulin sensitizer, insulin mimetic or insulin replacement compounds are at least one order of magnitude lower than the concentrations needed to mimic insulin action when the vanadium (V)/VI) compounds are used alone. From a practical point of view, the toxicity of these drugs is much lower than the toxicity of a drug based on vanadium compounds alone. This represents an important advantage of the (pharmaceutical) and pharmaceutical compositions of the present invention in respect of the compositions known in the art, for the treatment and/or prevention of diabetes mellitus.

Compounds of Formula I or Formulas II and IIA, and vanadium salts thereof are useful as pharmaceutical agents, and can be provided as pharmaceutical compositions. The pharmaceutical compositions can be manufactured in a manner that is itself known, e.g., by means of a conventional mixing, dissolving, granulating, dragee-making, levigating, emulsifying, encapsulating, entrapping or lyophilizing processes.

Pharmaceutical compositions can be formulated in conventional manner using one or more physiologically acceptable carriers comprising excipients and auxiliaries that facilitate processing of the active compounds into preparations that can be used pharmaceutically. Proper formulation is dependent upon the route of administration chosen.

For injection, the compounds prepared according to the methods of the invention can be formulated in appropriate aqueous solutions, such as physiologically compatible buffers such as Hanks's solution, Ringer's solution, or physiological saline buffer. For transmucosal and transcutaneous administration, penetrants appropriate to the barrier to be permeated are used in the formulation. Such penetrants are generally known in the art.

For oral administration, the compounds can be formulated readily by combining the active compounds with pharmaceutically acceptable carriers well-known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained with solid excipient, optionally grinding a resulting mixture, and processing the mixture of granules, after adding suitable auxiliaries, if desired, to obtain tablets or dragee cores. Suitable excipients are, in particular, fillers such as cellulose preparations such as, for example, maize starch, wheat starch, rice starch, potato starch, gelatin, gum tragacanth, methyl cellulose, hydroxypropylmethylcellulose, sodium carboxymethylcellulose, and/or polyvinylpyrrolidone (PVP). If desired, disintegrating agents can be added, such as the cross-linked polyvinyl pyrrolidone, agar, or alginic acid or a salt thereof such as sodium alginate. Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration. For buccal administration, the compositions can take the form of tablets or lozenges formulated in conventional manner.

For administration by inhalation, the compounds prepared according to the methods of the invention are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multi-dose containers, with an added preservative. The compositions can take such forms as suspensions, solutions or emulsions in oily or aqueous vehicles, and can contain formulatory agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyloleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use. The compounds can also be formulated in rectal compositions such as suppositories or retention enemas, e.g., containing conventional suppository bases such as cocoa butter or other glycerides.

In addition to the formulations described previously, the compounds can also be formulated as a depot preparation. Such long acting formulations can be administered by implantation (for example subcutaneously or intramuscularly) or by intramuscular injection. Thus, for example, the compounds can be formulated with suitable polymeric or hydrophobic materials (for example as an emulsion in acceptable oil) or ion exchange resins, or as sparingly soluble derivatives, for example, as a sparingly soluble salt.

A pharmaceutical carrier for hydrophobic embodiments of the antidiabetic, insulin sensitizer, insulin mimetic or insulin replacement compounds of the invention is a cosolvent system comprising benzyl alcohol, a nonpolar surfactant, a water-miscible organic polymer, and an aqueous phase. The cosolvent system can be the VPD co-solvent system. VPD is a solution of 3% w/v benzyl alcohol, 8% w/v of the nonpolar surfactant polysorbate 80, and 65% w/v polyethylene glycoL300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system can be varied considerably without destroying its solubility and toxicity characteristics. Furthermore, the identity of the co-solvent components can be varied: for example, other low-toxicity nonpolar surfactants can be used instead of polysorbate 80; the fraction size of polyethylene glycol can be varied; other biocompatible polymers can replace polyethylene glycol, e.g. polyvinyl pyrrolidone; and other sugars or polysaccharides can substitute for dextrose.

Alternatively, other delivery systems for hydrophobic pharmaceutical compounds can be employed. Liposomes and emulsions are well known examples of delivery vehicles or carriers for hydrophobic drugs. Certain organic solvents such as dimethylsulfoxide also can be employed, although usually at the cost of greater toxicity. Additionally, the compounds can be delivered using a sustained-release system, such as semipermeable matrices of solid hydrophobic polymers containing the therapeutic agent. Various sustained-release materials have been established and are well known by those skilled in the art. Sustained-release capsules can, depending on their chemical nature, release the compounds for a few weeks up to over 100 days. Depending on the chemical nature and the biological stability of the therapeutic reagent, additional strategies for protein and nucleic acid stabilization can be employed.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions of the compounds prepared according to the methods of the invention can be formulated and administered through a variety of means, including systemic, localized, or topical administration. Techniques for formulation and administration can be found in “Remington's Pharmaceutical Sciences,” Mack Publishing Co., Easton, Pa. The mode of administration can be selected to maximize delivery to a desired target site in the body. Suitable routes of administration can, for example, include oral, rectal, transmucosal, transcutaneous, or intestinal administration; potential delivery, including intramuscular, subcutaneous, intramedullary injections, as well as intrathecal, direct intraventricular, intravenous, intraperitoneal, intranasal, or intraocular injections.

Pharmaceutical compositions suitable for use include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For administration to non-human animals, the drug or a pharmaceutical composition containing the drug may also be added to the animal feed or drinking water. It will be convenient to formulate animal feed and drinking water products with a predetermined dose of the drug so that the animal takes in an appropriate quantity of the drug along with its diet. It will also be convenient to add a premix containing the drug to the feed or drinking water approximately immediately prior to consumption by the animal.

Preferred compounds prepared according to the methods of the invention will have certain pharmacological properties. Such properties include, but are not limited to oral bioavailability, low toxicity, low serum protein binding and desirable in vitro and in vivo half-lives. Assays may be used to predict these desirable pharmacological properties. Assays used to predict bioavailability include transport across human intestinal cell monolayers, including Caco-2 cell monolayers. Serum protein binding may be predicted from albumin binding assays. Such assays are described in a review by Oravcovà et al. (1996, Journal of Chromatography B-Biomedical Applications 677:1-28). Compound half-life is inversely proportional to the frequency of dosage of a compound. In vitro half-lives of compounds may be predicted from assays of microsomal half-life as described by Kuhnz and Gieschen (1998, Drug Metabolism and Disposition 26:1120-1127).

Toxicity and therapeutic efficacy of such compounds can be determined by conventional pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds that exhibit high therapeutic indices are preferred. The data obtained from these cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition. (See, e.g. Fing et al., 1975, in THE PHARMACOLOGICAL BASIS OF THERAPEUTICS, Ch. 1, p. 1).

Dosage amount and interval can be adjusted individually to provide plasma levels of the active moiety that are sufficient to maintain bacterial cell growth-inhibitory effects. Usual patient dosages for systemic administration should be kept so that less than 1.5 mg equivalents of vanadium is administered per day to a 70 kg man. In cases of local administration or selective uptake, the effective local concentration of the compound cannot be related to plasma concentration.

In a further embodiment the present invention pharmaceutical compositions for the treatment of diabetes, particularly for diabetes mellitus, in particular for insulin resistant states, e.g. diabetes type 2 or diseases related thereto, are provided. Preferably, the (pharmaceutical) composition according to the invention is an antidiabetic medicament. Pharmaceutical compositions according to the present invention typically comprise the inventive combination of arylalkylamines and pharmaceutically acceptable vanadium compounds. The inventive pharmaceutical compositions are suitable for the treatment of diabetes in prior, simultaneous or subsequent administration. Typically, these pharmaceutical compositions are administered orally or parenterally, including the intravenous, intramuscular, intraperitoneal, subcutaneous, rectal and topical routes of administration.

Toxicity and therapeutic efficacy of the inventive (pharmaceutical) composition comprising arylalkylamines and pharmaceutically acceptable vanadium (V)/VI), (VI) compounds may be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, which may be expressed as the ratio LD₅₀/ED₅₀. Inventive compounds (or compositions) which exhibit large therapeutic indices are preferred. While compounds that potentially exhibit toxic side effects may be used, care should be taken to design a delivery system that targets such reagents to the site of affected tissue in order to minimize potential damage to normal cells and, thereby, reduce side effects.

Data obtained from cell culture assays and animal studies can be used in formulating a range of dosage for use in humans. The dosage of the inventive compounds to be administered lies preferably within a range of circulating concentrations that include the ED₅₀ with little or no toxicity. The dosage may vary within this range depending upon the dosage form employed and the route of administration utilized. For any inventive compounds to be administered in the present invention, the therapeutically effective dose can be estimated initially from cell culture assays. A dose may be formulated in animal models to achieve a circulating plasma concentration range that includes the IC₅₀ (i.e., the concentration of the test compound which achieves a half-maximal inhibition of activity) as determined in cell culture. Such information can be used to more accurately determine useful doses in humans. The compounds of the invention preferably have an IC₅₀ less than 10 μM, which can be determined by biochemical or cellular assays typically used in the art. Some compounds of the invention are effective at concentrations of 1-10 mM. Based on these numbers, it is possible to derive an appropriate dosage for administration to subjects. Formation of prodrugs is well known in the art in order enhance the properties of the parent compound. Such properties include solubility, absorption, biostability and release time (see “Pharmaceutical Dosage Form and Drug Delivery Systems” (Sixth Edition), edited by Ansel et al., publ. by Williams & Wilkins, pgs. 27-29, (1995)). Commonly used prodrugs of the inventive compounds can be designed to take advantage of the major drug biotransformation reactions and are also to be considered within the scope of the invention. Major drug biotransformation reactions include N-dealkylation, O-dealkylation, aliphatic hydroxylation, aromatic hydroxylation, N-oxidation, S-oxidation, deamination, hydrolysis reactions, glucuronidation, sulfation and acetylation (see Goodman and Gilman's The Pharmacological Basis of Therapeutics (Ninth Edition), editor Molinoff et al., publ. by McGraw-Hill, pages 11-13, (1996)). The pharmaceutical compositions according to the present invention can be prepared such that they may be administered orally, parenterally, nasally, sublingually, rectally or vaginally. Parenteral administration included intravenous, intraarticular, intramuscular, intraperitoneal, and subcutaneous injections, as well as use of infusion techniques. One or more compounds of the invention may be present in association with one or more non-toxic pharmaceutically acceptable ingredients and optionally, other active anti-proliferative agents, to form the inventive pharmaceutical composition. These compositions can be prepared by applying known techniques in the art suck as those taught in Remington's Pharmaceutical Sciences (Fourteenth Edition), Managing Editor, John E. Hoover, Mach Publishing Co., (1970) or Pharmaceutical Dosage Form and Drug Delivery Systems (Sixth Edition), edited by Ansel et al., publ. by Williams & Wilkins, (1995).

As indicated above, pharmaceutical compositions containing the inventive combination of arylalkylamines and pharmaceutically acceptable vanadium (V)/VI) compounds may be in a form suitable for oral use. Dosage requirements are dependent e.g. upon the age, the body weight, gender, the method of administration (e.g. orally or parenterally) and the severity of the disease to be treated.

Methods for formulating the pharmaceutical compositions are provided as set forth herein and adapted to the specific mode of administration.

Methods for treating diabetes mellitus, type I or type II, are herein provided, comprising administering to a animal, most preferably a human in need thereof a therapeutically-effective amount of a compound or vanadium, selenium, molybdenum or tungsten salt thereof, and pharmaceutical composition thereof, of the invention.

Throughout the description and claims the word “comprise” and variations of the word, are not intended to exclude other technical features, additives, components, or steps. The abstract of this application is incorporated herein as reference. Additional objects, advantages and features of the invention will become apparent to those skilled in the art upon examination of the description or may be learned by practice of the invention. The following examples and drawings are provided by way of illustration, and they are not intended to be limiting of the present invention

The following Examples are provided for the purposes of illustration and are not intended to limit the scope of the present invention. The present invention is not to be limited in scope by the exemplified embodiments, which are intended as illustrations of individual aspects of the invention. Indeed, various modifications of the invention in addition to those shown and described herein will become apparent to those skilled in the art from the foregoing description and accompanying drawings. Such modifications are intended to fall within the scope of the appended claims.

### EXAMPLES

The following materials and methods are used in the specific Examples set forth below.

Materials

2-[1,2-³H]-D-deoxyglucose (26 Ci/mmol) was obtained from PerkinElmer Life and Analytical Sciences Products (Boston, Mass.) and [¹⁴C]Benzylamine (59 Ci/mmol) was obtained from Amersham Biosciences (Little Chalfont Buckinghamshire, England). Purified porcine insulin was a kind gift from Eli Lilly (Indianapolis, Ind.). Semicarbazide hydrochloride, benzylamine hydrochloride, sodium orthovanadate, wortmannin and other chemicals were purchased from Sigma Aldrich (St. Louis, Mo.). LY294002 was purchased from Calbiochem (San Diego, Calif.). Ketamine was obtained from Mérieux (Imalgene, Mérieux, France). Collagenase type I was obtained from Worthington (Lakewood, N.J.) and collagenase P from Roche Diagnostics (Basel, Switzerland). The osmotic minipumps used in chronic studies were from Alza Corporation (Palo Alto, Calif.). All electrophoresis reagents and molecular weight markers were obtained from Bio-Rad. Enhanced chemiluminescence reagents (super signal substrate) were from Amersham (Arlington Heights, Ill.). Anti-phospho-tyrosine monoclonal antibody and anti-insulin receptor β-chain polyclonal antibodies were purchased from BD Biosciences (Franklin Lakes, N.J.). Anti-phospho-Thr308-PKB and anti phosho-Ser473—PKB polyclonal antibodies were purchased from Cell Signaling Technologies (Beverly, Mass.).

Fmoc-Rink linker and solid supports were supplied by Calbiochem-Novabiochem AG. DIPCDI was obtained from Fluka Chemika (Buchs, Switzerland) and HOBt from Albatross Chem Inc. (Montreal, Canada). Solvents for peptide synthesis and RP-HPLC were obtained from SDS (Barcelona, Spain). Trifluoroacetic acid was supplied by KaliChemie (Bad Wimpfen, Germany). Semicarbazide hydrochloride, benzylamine hydrochloride, hydrogen peroxide, horseradish peroxidase and other chemicals were purchased from Sigma Aldrich (St. Louis, Mo., USA). Purified human VAP-1 was a kind gift from BioTie Therapeutics (Turku, Finland). Amplex red reagent (10-acetyl-3,7-dihydroxyphenoxazine) was from Molecular Probes (Eugene, Oreg., USA). Other chemicals were obtained from Aldrich (Milwaukee, Wis.) and were of the highest purity grade available. All commercial reagents and solvents were used as received. HPLC was performed using an Alliance 2795 Waters Chromatography system with a reverse-phase column C₁₈ X-Terra 5 μm 4.6×100 mm with UV detection at 220 and 254 nm. Mass spectra were recorded on a Waters Alliance HT 2795 system with Dual □Absorbance detector 2487 and Micromass ZQ Mass Spectrometer. IR were performed by Thermo Nicolet FT-IR Nexus spectrometer 4000-400 cm⁻¹ range. Solid-phase reactions were performed in polypropylene syringes fitted with a polyethylene porous disc. Solvents and soluble reagents were removed by filtration. Solvents and soluble reagents were removed by filtration. The purity of the arylalkylamines synthesized was determined by HPLC using a C₁₈ X-Terra 5 μm 4.6×100 mm column with linear gradient 0% B-100% B in 10 min (A: 0.1 TFA % in H₂O, B: 0.1 TFA % in ACN, 1 mL/min) with UV detection at 220/254 nm. All compounds were characterized by HPLC-MS.

Animals

Male Wistar rats weighting 180-220 g were purchased from Harlan (Interfauna Ibèrica S.A., Spain). Diabetes was induced by a single intraperitoneal injection of a freshly prepared solution of streptozotocin (in some studies the dose was 45 mg/kg body weight and in some others 100 mg/kg body weight dissolved in 50 mM citrate buffer, pH 4.5). Only diabetic animals with glycemia above 300 mg/dl were used. The animals were housed in animal quarters at 22° C., with a 12 h light/12 h dark cycle and were fed ad libitum. All procedures used were approved by the animal ethical committee of the University of Barcelona, Spain. Male mice C57 BL/Ks bearing the db/db mutation (Jackson Laboratories, Bar Harbor, Me.) were purchased from Harlan France (Gannat, France). C57BL/6J male mice were assigned for 16 weeks to very high-fat diet containing (in kcal): 72% from fat, 28% from proteins and <1% from carbohydrates (Burcelin et al., 2002, Am. J. Physiol. Endocrinol. Metab 282: E834-E842).

Chronic Treatments of Diabetic Animals

Osmotic minipumps delivering B6V10 (2.5 μmol/kg body wt/day) or decavanadate (2.5 μmol/kg body wt/day) were implanted subcutaneously in diabetic rats anaesthetised by ketamine hydrochloride (95 mg/kg) and xylasine (10 mg/kg). Animals that did not receive B6V10 or decavanadate were sham-operated. Glycemia was measured on arterio-venous blood collected from the tail vessels at 09:00 am for two weeks, before the administration of vanadate. Insulin concentrations were determined before and after treatment. In another set of experiments, B6V10 was orally administered at a single dose of 5 μmol/kg/day during the first week and 10 μmol/kg/day during 2 additional weeks by gastric gavage. A control group received the corresponding decavanadate salt in the absence of benzylamine. At the end of the treatment, animals were sacrificed and the liver, fat pad, heart and lung were kept at −80° C. and the plasma at −20° C. until their use for in vitro analysis.

Amine Oxidase Activity Assays

The continuous spectrophotometric detection of SSAO-dependent H₂O₂ production based on a peroxidase-coupled reaction was performed as previously described by Abella et al. (2004, Diabetologia 47: 429-438) and following the procedure described by Holt et al., (1997, Anal. Biochem. 244: 384-392).

Analytical Methods

In glucose tolerance tests and in chronic treatments, circulating glucose concentrations were determined by a rapid glucose analyser (Accutrend® Sensor Comfort, Roche, Basel. Switzerland). Plasma insulin (IRI) concentration was determined by ELISA method using a kit obtained from Crystal Chem Inc. (Downers Grove, Ill.). Plasma triglycerides (Biosystems, Barcelona, Spain) and NEFAS (Wako Chemicals, Neuss, Germany) were determined with standard calorimetric methods.

Analysis of Intracellular Signaling

Isolated fat cells were disrupted for total membrane preparation by hypo-osmotic lysis in a 20 mM HES buffer and an antiprotease and antiphosphatase cocktail as reported by Abella et al. (2003, Diabetes 52: 1004-1013). Protein concentrations were determined by the Bradford method (Bradford, 1976, Anal. Biochem. 72: 248-254) with gamma-globulin as protein concentration standard. Immunoprecipitation and immunoblot assays were performed as previously described by Abella et al. (2004, Id.) with the use of a monoclonal antiphosphotyrosine antibody for the immunoprecipitation and an anti-insulin receptor antibody for immunoblotting, respectively. SDS-polyacrylamide gel electrophoresis was performed on membrane proteins following conventional procedures. Proteins were transferred to Immobilon and immunoblotting was performed as reported by Castello et al. (1994, J. Biol. Chem. 269: 5905-5912).

Calculations and Statistical Analysis

Insulin and glucose responses during the glucose tolerance test were calculated as the incremental plasma values integrated over a period of 120 min after injection of glucose. Areas under curves of insulin and glucose responses were calculated using the Graph Prism program (Graphpad Software, Inc., San Diego, Calif.). Data distribution was analyzed by the Kolmogorov-Smirnov test. Data were presented as mean±SEM and unpaired

Student's t test was used to compare two groups. When experimental series involved more than two groups, statistical analysis was done by one-way or two-way ANOVA and further post-hoc (Dunnett, Tukey or Bonferroni) t tests. Statistical analysis was performed with SPSS 11.0 or GraphPad Prism 4 programs.

In the accompanying examples the following abbreviations have been used: ACN, acetonitrile; TFA, trifluoroacetic acid; THF, tetrahydrofuran; MeOH, methanol; Fmoc, 9-fluorenylmethyloxycarbonyl; DMF, dimethylformamide; DIEA, N,N-diisopropylethylamine; TMOF, trimethylorthoformate; MEI, methyl iodide; XAL, ((9-(amino)xanthen-2-yl)oxy)butanoic acid handle; MBHA, p-methylbenzhydrylamine resin; Rink resin, 4-(2′,4′-dimethoxyphenyl-Fmoc-aminomethyl)-phenoxy resin; ESI-MS, Electrospray ionization mass spectroscopy; IR, infrared spectroscopy; HPLC, high performance liquid chromatography; t_{R}, retention time; NMR, nuclear magnetic resonance.

### Example 1

### Preparation of hexaquis(benzylammonium) decavanadate (V) dihydrate(C₇NH₁₀)₆(V₁₀O₂₈)H₂O)₂

Sodium vanadate (1.120 g, 10 mol) was dissolved in 30 ml of water and pH was adjusted at 7.4 by addition of hydrochloric acid 1N. Then, benzylammonium hydrochloride was added (0.525 g, 6 mol) which produced the precipitation of a solid. It was filtered under vacuum and dried over phosphorus pentoxide in a vacuum oven at 30° C. to obtain the compound of the title. Afterwards, a saturated solution of said compound was prepared, and evaporated to the ambient to obtain the compound of the title as crystals. Expected (C₄₂H₆₄N₆O₃₀V₁₀): C, 30.71%; H, 3.93%; N, 5.12%; O 29.22%; V, 31.02%. Found (C₄₂H₆₄N₆O₃₁V₁₀): C, 30.59%; H, 3.89%; N, 4.87%; O 29.94%; V, 30.71%. IR (ATR, νₘₐₓ/cm⁻¹): 3033 mν (—NH3), 1566 δw (—NH3), 973 w, 943 s, 918 s, 832 m, 790 w, 751 m, 699 m. ¹H-RMN (DMSO-d₆): 7.4 (m, 5H), 3.9 (s, 2H) ppm. ¹³C-RMN (DMSO-d₆): 44 (CH₂), 128 (CH₂), 130 (4CH) ppm. ⁵¹V-RMN (78.87 MHz, DMSO-d₆): −415.1, −489.9, −508.8 ppm.

### Example 2

### Preparation of pentaquis(benzylammonium) decavanadate (V) dihydrate (C₇NH₁₀)₅(HV₁₀O₂₈)(H₂O)₂

Sodium vanadate (1.120 g, 10 mol) was dissolved in 30 ml of water and pH was adjusted at 5.5 by addition of hydrochloric acid 1N. Then, benzylammonium hydrochloride was added (0.525 g, 6 mol) which produced the precipitation of a solid. The work up was identical to the one described for the compound of the Example 1. Expected (C₃₅H₅₁N₅O₂₈V₁₀): C, 28.04%; H, 3.43%; N, 5.23%; O, 33.98%; V, 33.98%. Found (C₄₀H₆₂N₅O₃₀V₁₀): C, 28.91%; H, 3.78%; N, 4.98%; O, 29.23%; V, 33.1%. IR (ATR, νₘₐₓ/cm⁻¹): 3033 mν (—NH3), 1566 δw (—NH3), 972 w, 945 s, 918 s, 832 m, 747 w, 700 m. ¹H-RMN (DMSO-d₆): 7.4 (m, 5H), 3.9 (s, 2H) ppm. ¹³C-RMN (DMSO-d₆): 44 (CH₂), 128 (CH₂), 130 (4CH) ppm. ⁵¹V-RMN (78.87 MHz, DMSO-d₆): −417, −491.5, −510.0 ppm.

### Example 3

### Preparation of Tetraquis(Benzylammonium) Decavanadate (V) Dihydrate (C₇NH₁₀)₄(H₂V₁₀O₂₈)H₂O)₂

Sodium vanadate (1.120 g, 10 mol) was dissolved in 30 ml of water and pH was adjusted at 2 by addition of hydrochloric acid 1N. Then, benzylammonium hydrochloride was added (0.525 g, 6 mol) which produced the precipitation of a solid. The work up was identical to the one described for the compound of the Example 1. Expected (C₂₈H₄₂N₄O₂₈V₁₀): C, 24.16%; H, 3.04%; N, 4.02%; O, 29.22%; V, 32.18%. Found (C₃₂H₅₄N₄O₃₉V₁₀): C, 23.04%; H, 3.27%; N, 3.79%; O, 37.71%; V, 32.19%. IR (ATR, νₘₐₓ/cm⁻¹): 3033 mν (—NH3), 1566 δw (—NH3), 972 w, 939 s, 824 m, 750 w, 701 w, 644 m. ¹H-RMN (DMSO-d₆): 7.4 (m, 5H), 3.9 (s, 2H) ppm. ¹³C-RMN (DMSO-d₆): 44 (CH₂), 128 (CH₂), 130 (4CH) ppm. ⁵¹V-RMN (78.87 MHz, DMSO-d₆): -407.5, −491.8, −512.1 ppm.

### Example 4

### Preparation of 2-hydroxy-3-methoxybenzylamine

2-hydroxy-3-methoxybenzaldehyde (1.52 g, 10 mmol) was dissolved in methanol (10 ml). A solution of hydroxylamine in water at 50% (2.4 ml, 40 mmol) was added and the mixture was refluxed. After 30 min, the solvent was distilled under reduced pressure and the resulting solid was dissolved in ether, washed firstly with two volumes of saturated HCl 1N, then with saturated NaHCO₃ solution, and finally with saturated NaCl solution. The organic phase was dried with MgSO₄, and concentrated at reduced pressure to give the 2-hydroxy-3-methoxybenzoxime.

2-hydroxy-3-methoxybenzoxime (83.5 mg, 0.5 mmol) and PtO₂.1H₂O (5.67 mg, 0.022 mmol) was dissolved in acetic acid (10 ml). The mixture was pressurized with H₂ to 4 bars and reacted overnight. The mixture was filtered through celite and the acetic acid was distilled under reduced pressure. The crude was washed with tetrahydrofuran (R: 85%/R: 95%). ESI-MS: M_{calc} (C₈H₁₁O₂N): 153.8, [M+1]⁺: 154.8 m/z, [M-NH₃]⁺: 136.7 m/z. ¹H-RMN (400 MHZ, CDCl₃): 3.7 (CH₃), 4.0 (CH₂), 7.0 (m, 3H) ppm. ¹³C-RMN (400 MHz, CDCl₃): 38, 54, 111, 118, 120 ppm. HPLC: t_{R}: 3.62 min, H₂O (TFA 1%)/ACN (TFA 1%) 0-100% in 15 min, column C₁₈ Symmetry 300 5 μm (3.9×150), λ=220/254 nm.

### Example 5

### Preparation of 1-aminomethyl-n-naphthalene-2-ol

2-hydroxynaphthalene-1-carbaldehyde, (1.52 g, 10 mmol) was dissolved in methanol (10 ml). A solution of hydroxylamine in water at 50% (2.4 ml, 40 mmol) is added and the mixture was refluxed. After 30 min, the solvent was distilled under reduced pressure and the resulting solid was dissolved in ether, washed firstly with two volumes of saturated HCl 1N, then with saturated NaHCO₃ solution, and finally with saturated NaCl solution. The organic phase was dried with MgSO₄, and concentrated at reduced pressure to give the oxime of 2-hydroxy-naphthalene-1-carbaldehyde.

The oxime of 2-hydroxy-naphthalene-1-carbaldehyde (94 mg, 0.5 mmol) and PtO₂.1H₂O (5.67 mg, 0.022 mmol) was dissolved in acetic acid (10 ml). The mixture was pressurized with H₂ to 4 bars and reacted during all night. It was filtered through celite and the acetic acid was distilled under reduced pressure. The crude was washed with tetrahydrofuran. (R: 94%/P: 90%). ESI-MS: M_{calc} (C₈H₁₁O₂N): 173.8, [M+1]⁺: 174.8 m/z, [M-NH₃]⁺: 157.7. ¹H-RMN (400 MHz, CDCl₃): 4.3 (CH₂), 7.2 (CH), 7.3 (CH), 7.7 (CH), 8.0 (CH) ppm. ¹³C-RMN (400 MHz, CDCl₃): 38, 111, 122, 124, 128, 132, 134, 138, 160 ppm. HPLC: t_{R}: 4.4 min, H₂O (TFA 1%)/ACN (TFA 1%) 0-100% in 15 min, column C₁₈ Symmetry 300 5 μm (3.9×150), λ=220/254 nm.

### Example 6

### Preparation of 2-hydroxy-3-methoxybenzylamine

The title compound was obtained by solid-phase synthesis. Solid-phase manipulations were performed in polypropylene syringes fitted with a polyethylene porous disc. Solvents and soluble reagents were removed by filtration. 2-hydroxy-3-methoxybenzaldehyde (15 equiv, 250 mg) was condensed on a Rink resin (100 mg, 1.1 mmol/g) using TMOF (1 ml) as a solvent and the mixture was stirred overnight under Ar at 25° C. to give the aldimine. The resin was filtered off and washed with trimethyl orthoformate (TMOF; 5×1 min) and dry tetrahydrofuran (THF; 5×1 min). Then, the reduction of the imine was carried out with LiBH₄ (15 equiv, 40 μl) in dry THF under Ar and the mixture was stirred for 5 h at 65° C. to give the corresponding secondary amine. The resin was filtered off and washed several times with THF, H₂O, MeOH and DCM. The amine was cleaved from the resin with 5% TFA in DCM for 5 h at 25° C. The solution was filtered and evaporated under N₂ to dryness and the compound of the title was obtained with 98% of purity. MS: [(M+H)⁺_{calc.} 153, (M+H)⁺ₑₓₚ 154.0 m/z] and [(M-NH₃)⁺ₑₓₚ 137.0 m/z]. HPLC: t_{R}: 3.9 min., H₂O (0.1% TFA)/ACN (0.1% TFA) 0-100% in 15 min, column C₁₈ Symmetry 300 5 μm (3.9×150), λ=220/254 nm.

### Example 7

### Preparation of 2-hydroxynaphthylmethylamine

The title compound was obtained following a procedure analogous to the one described in Example 6 from 2-hydroxy-1-naphthaldehyde (15 equiv, 283 mg). Purity: 93%. ESI-MS: [(M+H)⁺_{calc} 173, (M+H)⁺ₑₓₚ 174 m/z] and [(M-NH₃)⁺ₑₓₚ 156.9 m/z]. HPLC: t_{R}: 5.3 min., H₂O (0.1% TFA)/ACN (0.1% TFA) 0-100% in 15 min, column C₁₈ Symmetry 300 5 μm (3.9×150), λ=220/254 nm.

### Example 8

### Preparation of 2,3-dimethoxybenzaldehyde (Intermediate for the Preparation of 2,3-dimethoxybenzylamine)

2-hydroxy-3-methoxybenzaldehyde (3.28 mmol, 500 mg) was dissolved in 20 ml DMF and then K₂CO₃ (3.28 mmol, 453 mg) was added. The mixture was stirred at room temperature for 15 min. Then, methyliodide (Mel; 3.28 mmol, 193 μL) was added and the mixture was stirred overnight at room temperature. Afterwards, it was concentrated under reduced pressure and then diluted with 50 ml of 5% NaOH aqueous solution. The modified aldehyde was extracted with DCM (3×50 ml), dried with MgSO₄ and concentrated under reduced pressure. ESI-MS: [(M+H)⁺_{calc}:166.1, (M+H)⁺ₑₓₚ: 167.8 m/z]. ¹H-RMN (400 MHz, CDCl₃): 3.73 (2(OCH3)), 7.0 (m, 3H) ppm. ¹³C-RMN (400 MHz, CDCl₃): 56, 112, 118, 120, 124, 130, 154 ppm. HPLC: t_{R}: 8.1 min, gradient H₂O (0.1% TFA)/ACN(0.1% TFA) 0-100% in 15 min, column C₁₈ Symmetry 300 5 μm (3.9×150), λ=220/254 nm.

### Example 9

### Preparation of 2,3-dimethoxybenzylamine

Solid-phase manipulations were performed in polypropylene syringes fitted with a polyethylene porous disc. Solvents and soluble reagents were removed by filtration. 2,3-dimethoxybenzaldehyde (15 equiv) was condensed on a H₂N-XAL-MBHA resin (38 mg, 0.7 mmol/g) using TMOF (1 ml) as a solvent and the mixture was stirred overnight under Ar at 25° C. to give the aldimine. Then, the resin was filtered off and washed with TMOF (5×1 min.) and dry THF(5×1 min.). The reduction was carried out with LiBH₄ (15 equiv, 40 μl) in dry THF under Ar and the mixture was stirred for 5 h at 65° C. to give the corresponding secondary amine. The resin was filtered off and washed several times with THF, H₂O, MeOH and DCM. The amine was cleaved from the resin with 5% TFA in DCM for 5 h at 25° C. The solution was filtered and evaporated under N₂ to dryness. ESI-MS: [(M+H)⁺_{calc} 167.2, (M+H)⁺ₑₓₚ 167.7], and [(M-NH3)⁺ₑₓₚ 150.7]. HPLC: t_{R}: 4.3 min, H₂O (0.1% TFA)/ACN (0.1% TFA) 0-100% in 15 min, column C₁₈ X-Terra 15 μm (4.6×10), λ=220/254 nm.

### Example 10

### Preparation of 2-methoxynaphthylaldehyde

The compound of the title was obtained following a procedure analogous to the one described in Example 8 for 2-hydroxynaphthaldehyde. 2-hydroxynaphthaldehyde (2.90 mmol, 500 mg) was dissolved in 20 ml DMF and K₂CO₃ (2.90 mmol, 316 mg) was added. The mixture was stirred at room temperature for 15 min. Then, Mel (2.90 mmol, 147 μl) was added and the mixture was stirred overnight at room temperature. It was concentrated under reduced pressure and diluted with 50 ml of 5% NaOH aqueous solution. The modified aldehyde was extracted with DCM (3×50 ml), dried with MgSO₄ and concentrated under reduced pressure. ESI-MS: [(M+H)⁺_{calc}: 186.1, (M+H)⁺ₑₓₚ: 187.8 m/z]. ¹H-RMN (400 MHz, CDCl₃): 3.73 (OCH3), 7.2 (CH), 7.3 (CH), 7.7 (CH), 8.0(CH) ppm. ¹³C-RMN (400 MHz, CDCl₃): 56, 111, 122, 124, 128, 132, 134, 138, 160 ppm. HPLC: t_{R}: 10.2 min, gradient H₂O (0.1% TFA)/ACN (0.1% TFA) 0-100% in 15 min, column C₁₈ Symmetry 300 5 μm (3.9×150), λ=220/254 nm.

### Example 11

### Preparation of 2-methoxynaphthylamine

Solid-phase manipulations were performed in polypropylene syringes fitted with a polyethylene porous disc. Solvents and soluble reagents were removed by filtration. 2-methoxynaphthaldehyde (15 equiv) was condensed on a H₂N-XAL-MBHA resin (29 mg, 0.7 mmol/g) using TMOF (1 ml) as a solvent and the mixture was stirred overnight under Ar at 25° C. to give the aldimine. The resin was filtered off and washed with TMOF (5×1 min.) and dry THF (5×1 min.). Then the reduction was carried out with LiBH₄ (15 equiv, 40 μl) in dry THF under Ar and the mixture was stirred for 5 h at 65° C. to give the corresponding secondary amine. The resin was filtered off and washed several times with THF, H₂O, MeOH and DCM. The amine was cleaved from the resin with 5% TFA in DCM for 5 h at 25° C. The solution was filtered and evaporated under N₂ to dryness. ESI-MS: [(M+H)⁺_{calc} 187.2, (M+H)⁺ₑₓₚ: 187.7, (M-NH3)⁺ₑₓₚ: 170.3]. HPLC: t_{R}: 5.4 min, H₂O (0.1% TFA)/ACN (0.1% TFA) 0-100% in 15 min, column C₁₈ X-Terra 15 μm (4.6×10), λ=220/254 nm.

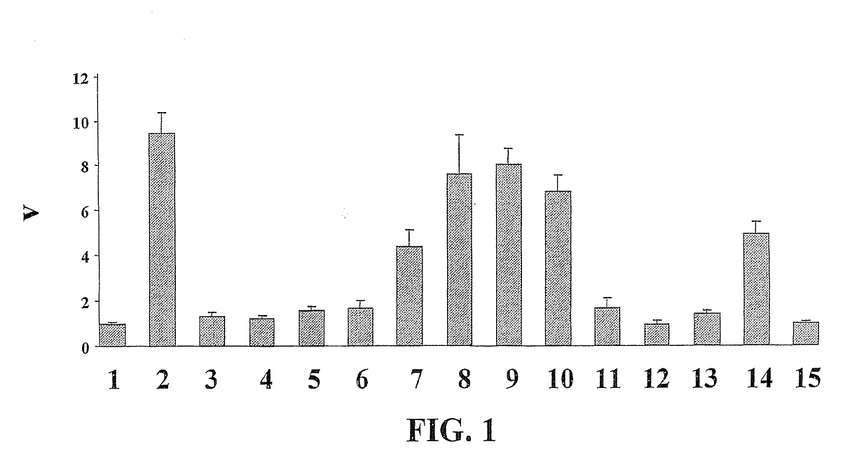

Examples 12-19 illustrate that the arylalkylamine vanadium (V) salts of the present invention are insulin mimickers better than those produced by the combination of vanadium plus amine, which is also illustrated in FIG. 1. They produce an increased glucose transport in the same way as insulin does. Example 13 also illustrates that chronic administration of benzylammonium decavanadate salts produces a rapid reduction of the hyperglycemia. Example 14 also shows that they can be taken orally.

### Example 12

### Effects of hexaquis(benzylammonium) Decavanadate pentaquis(benzylammonium) decavanadate and tetraquis(benzylammonium) decavanadate on Glucose Transport in Isolated Adipocytes

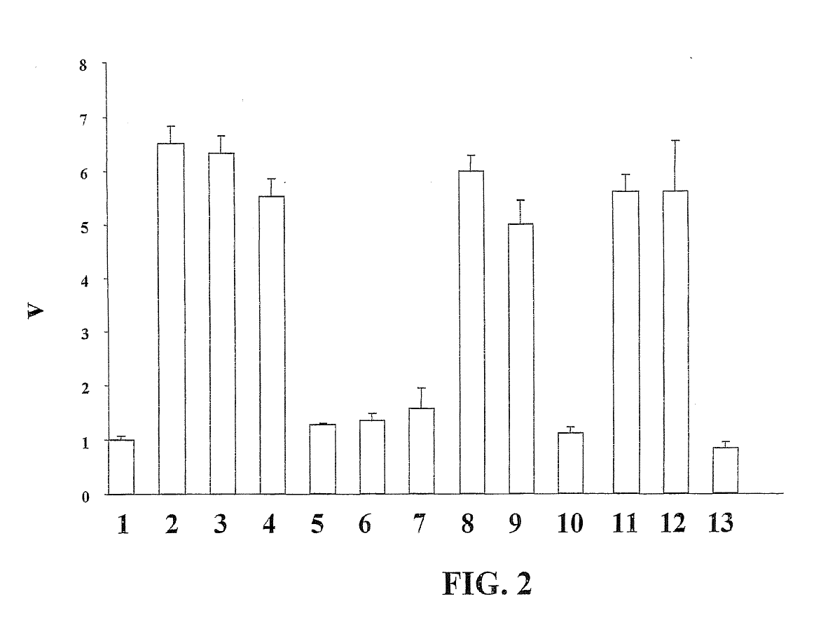

In order to determine whether salts formed by vanadate and benzylamine could produce an insulin mimetic effect, the effect of increasing concentrations of hexaquis(benzylammonium) decavanadate on glucose transport activity in isolated rat adipocytes was tested. The results of these experiments are shown in FIG. 1. Hexaquis(benzylammonium) decavanadate induced the stimulation of glucose transport which was perceptible from concentrations of 0.5 μM, with a maximal effect observed 2.5 μM and the semi-maximal effect above 1 μM. The stimulatory effect of the hexaquis(benzylammonium) decavanadate was completely blocked by semicarbazide, which indicates that the semicarbazide-sensitive amine oxidase activity is required for the effect. The maximum effect provoked by incubation with hexaquis(benzylammonium) decavanadate was greater than that produced by the presence of benzylamine and vanadate in combination (FIG. 1). These results indicated that hexaquis(benzylammonium) decavanadate is an insulin mimetic agent more powerful than the combination of vanadate and benzylamine. In similar assays, the activity of hexaquis(benzylammonium) decavanadate, pentaquis(benzylammonium) decavanadate and tetraquis(benzylammonium) decavanadate on glucose transport activity in isolated rat adipocytes were analyzed. The three compounds caused a pronounced stimulation on glucose transport (FIG. 2) and in the presence of the semicarbazide inhibitor this effect was inhibited.

### Example 13

### Effect of the Chronic Administration of hexaquis(benzylammonium) Decavanadate in Diabetic Rats

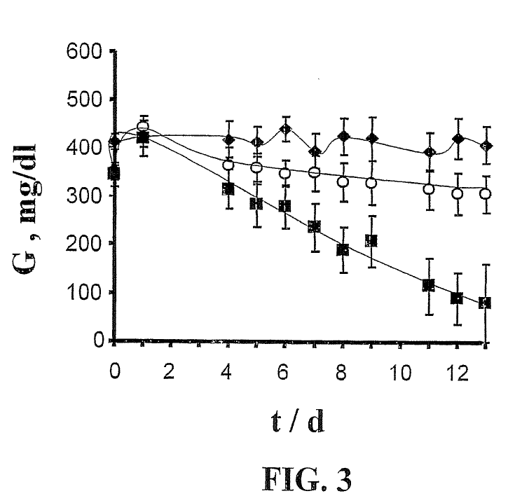

The effect of chronic administration of hexaquis(benzylammonium) decavanadate on glycemia from diabetic rats was determined. Thus, diabetes was induced in rats by intravenous administration of streptozotocin, which destroys the β-pancreatic cells that produce insulin. Treated rats with buffered solution used as solvent or with sodium decavanadate, did not modify substantially its glycemia during the two weeks of treatment (FIG. 3). Under these conditions, administration to the rats of hexaquis(benzylammonium) decavanadate produced a rapid reduction of the hyperglycemia that was detected after only four days of treatment (FIG. 3). After eleven days of treatment, glycemia in hexaquis(benzylammonium) decavanadate-treated rats was similar to the non-diabetic rats. At fourteen days of treatment, adipocytes from chronically hexaquis(benzylammonium) decavanadate-treated rats were isolated and glucose transport velocity determined; adipocytes of hexaquis(benzylammonium) decavanadate-treated rats showed an increased glucose transport under basal conditions equivalent to that seen in the presence of insulin. Moreover, an inverse correlation was detected between animal glycemia and basal glucose transport velocity, which suggested that adipocytes played a role in the antidiabetic effects of hexaquis(benzyl-ammonium) decavanadate.

### Example 14

### Effect of Oral and Chronic Administration of hexaquis(benzylammonium) Decavanadate in Diabetic Rats

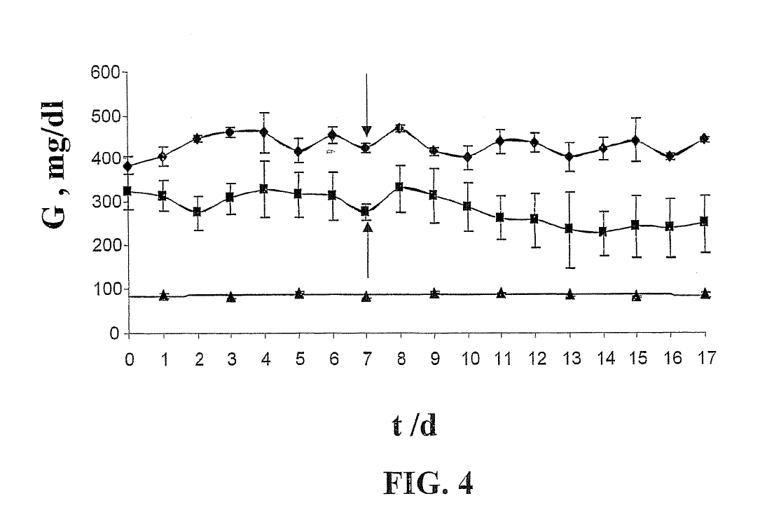

The effect of the oral administration of hexaquis(benzylammonium) decavanadate on glycemia from diabetic rats was also studied. Diabetes was induced in rats by intravenous administration of streptozotocin, and subsequently, a hexaquis(benzylammonium) decavanadate or sodium decavanadate unique dose was administered to the rats. Glycemia was not affected substantially in sodium decavanadate-treated rats during the seventeen days of treatment (FIG. 4). Under these conditions, administration of a 5 μmol/kg/day dose of hexaquis(benzylammonium) decavanadate for seven days produced a moderate decrease of hyperglycemia that was detected after but two days of treatment (FIG. 4). After seven days of treatment, the dose was increased at 10 μmol/kg/day which was maintained for an additional ten days. The dosage increase produced an additional decrease in glycemia of the animals. Thus, glycemia in sodium decavanadate treated rats was approximately 450 mg/dl and glycemia of hexaquis(benzylammonium) decavanadate treated rats was approximately 250 mg/dl.

### Example 15

### Analysis of Insulin Mimetic Capacity of Certain Vanadium Arylalkylamines

To determine the possible insulin mimetic effect of certain vanadium arylalkylamines, the capacity of these compounds to enhance glucose transport in isolated rat adipocytes was tested. Adipose cells from Wistar rats were incubated for 45 minutes in basal conditions (Basal) or in the presence of 100 nM insulin (Ins), and different concentrations of. hexaquis(benzylammonium) decavanadate (B6V10) in the absence or in the presence of 1 mM semicarbazide (SCZ). Subsequently, 2-DG transport was measured over a 5 min. interval.

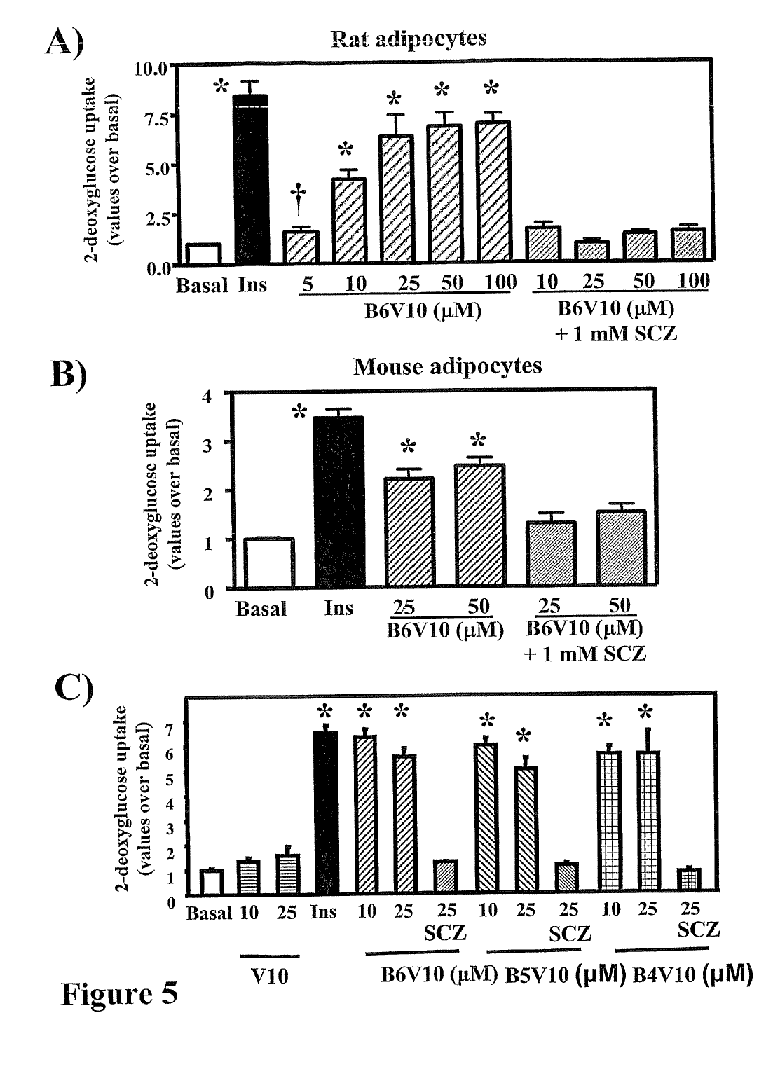

The results of these experiments are shown in FIG. 5A through 5C. B6V10 stimulated glucose transport in rat adipocytes in a concentration-dependent manner (FIG. 5A) and the maximal effect was 85% of the maximal stimulation caused by insulin. Notably, 25 μM B6V10 showed a greater stimulation of glucose transport than the combination of 100 μM benzylamine and 100 μM vanadate (data not shown). The stimulatory effect of B6V10 was completely blocked by semicarbazide, which indicates that SSAO activity is required to observe the effect of B6V10 in these cells. In contrast, sodium decavanadate salt (V10) alone at concentrations ranging from 5 to 50 μM did not stimulate glucose transport (data not shown; see FIG. 5C).

Similar stimulatory effects of B6V10 were detected in isolated mouse adipocytes (FIG. 5B). Adipose cells from FVB mice were incubated for 45 minutes in basal conditions (Basal) or in the presence of 100 nM insulin (Ins), and different concentrations of hexaquis(benzylammonium) decavanadate (B6V10) in the absence or in the presence of 1 mM semicarbazide (SCZ) and thereafter, 2-DG transport was measured over 5 min. The addition of benzylamine and V10 at equivalent concentrations showed no effect on glucose transport in isolated mouse adipocytes (data not shown), and stimulation of glucose transport by 100 μM B6V10 (93% increase) was greater than the stimulation that resulted from the combination of 1 mM benzylamine and 1 mM vanadate (51% increase). This result suggested that B6V10 has additional relevant biological properties compared to their combined components.

The effects of the three tested compounds (B6V10, B5V10 and B4V10) were compared. Adipose cells from Wistar rats were incubated for 45 minutes in basal conditions (Basal) or in the presence of 100 nM insulin (Ins), and different concentrations of. decavanadate (V10), hexaquis(benzylammonium) decavanadate (B6V10), pentaquis(benzylammonium) decavanadate (B5V10) or tetraquis(benzylammonium) decavanadate (B4V10) in the absence or in the presence of 1 mM semicarbazide (SCZ). 2-DG transport was measured over 5 min. intervals. All three compounds showed a similar potency as activators of glucose transport activity in isolated rat adipocytes (FIG. 5C). The stimulation of all three compounds on glucose transport was blocked in the presence of semicarbazide.

These results indicated that a lower ratio benzylamine/vanadium does not alter the insulin replacement potency of the arylalkylamine vanadium salts.

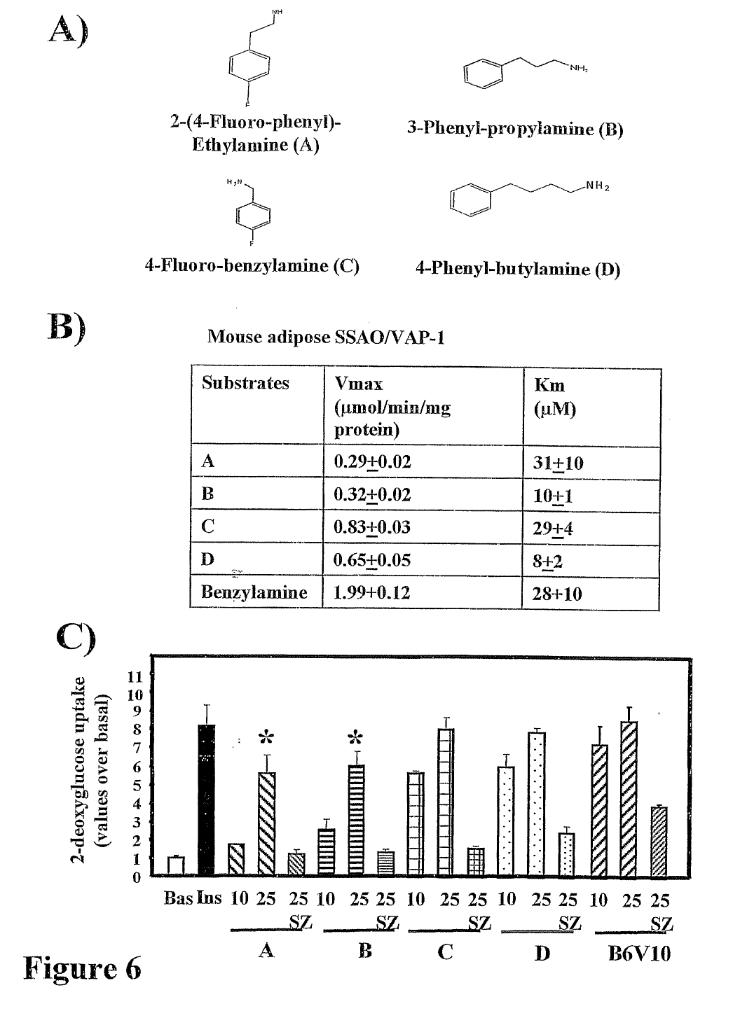

In additional experiments, compounds shown in FIG. 6A (named 2-(4-fluoro-phenyl)-ethylamine (compound A), 3-phenyl-propylamine (compound B), 4-fluoro-benzylamine (compound C) and 4-phenyl-butylamine (compound D)) were assessed using the experimental methods set forth above for the capacity to stimulate 2-DG uptake in isolated rat adipocytes. These results are shown in FIG. 6B. Adipose cells from Wistar rats were incubated for 45 minutes in basal conditions (Basal) or in the presence of 100 nM insulin (Ins), and different concentrations of vanadium salts of 2-(4-fluoro-phenyl)-ethylamine (compound A), 3-phenyl-propylamine (compound B), 4-fluoro-benzylamine (compound C) and 4-phenyl-butylamine (compound D). 2-DG transport was measured over 5 min. All four compounds markedly stimulated glucose transport of rat adipocytes and the maximal stimulatory effect was similar for the vanadium salts generated with compounds C and D. The stimulation induced at a low concentration (10 μM) was also higher for C— and D-derived vanadium salts than for A and B vanadium salts. These results indicated that arylalkylamine vanadium salts are a novel class of insulin replacement compounds.

### Example 16

Compounds listed in Table 1 were prepared according to the methods described above, purity was determined by HPLC at 220 nm, molecular weight calculated by mass spectrometer. Human and mouse SSAO activity was determined by detecting the production of hydrogene peroxide in the presence of the different compounds present at 1 mM for human and 100 μM for mice activity measurements. Compounds in Table 2″ were similarly assayed. Values are means of percentages relative to benzylamine-related hydrogen production. Kinetic Parameters for select compounds are set forth in Table 3 (Mouse SSAO) and Table 4 (Human SSAO).

TABLE 1

| | | | | | SSAO activity | |
|---|---|---|---|---|---|---|
| | | | | | (% relative to benzylamine^{b}) | |
| entry | Compounds | % Purity | MW_{calc} | MS_{found} | Human | Mouse |
| 1 | Benzylamine | — | — | — | 100 | 100 |
| 3 | (4-fluorophenyl)methanamine | 94 | 125.1 | 126.1 | 31.70 | 77.20 |
| 4 | p-tolylmethanamine | 90 | 121.1 | 105.1 | 0.00 | 0.00 |
| 5 | 4-ethylphenyl)methanamine | 99 | 135.2 | 136.0 | 0.00 | 17.30 |
| 6 | (4-tert-butylphenyl)methanamine | 82 | 149.2 | 150.0 | 0.00 | 1.00 |
| 7 | (4-isopropylphenyl)methanamine | 79 | 163.2 | 164.0 | 0.00 | 0.00 |
| 8 | (4-(trifluoromethyl)phenyl)methanamine | 98 | 175.1 | 159.0 | 0.50 | 83.00 |
| 9 | (4-(trifluoromethoxy)phenyl)methanamine | 80 | 191.1 | 192.0 | 0.00 | 66.20 |
| 10 | (2,4-dimethoxyphenyl)methanamine | 75 | 167.2 | 151.1 | 0.00 | 0.00 |
| 11 | (4-butylphenyl)methanamine | 72 | 163.2 | 147.1 | 0.00 | 27.00 |
| 13 | 4-(aminomethyl)-2-bromophenol | 91 | 202.1 | 201.9 | 0.00 | 0.00 |
| 14 | 2-(aminomethyl)-6-nitrophenol | 99 | 168.1 | 153.2 | 0.11 | 5.47 |
| 15 | biphenyl-4-yl-methanamine | 88 | 183.2 | 166.9 | 0.00 | 3.77 |
| 16 | (3-fluoro-4-methylphenyl)methanamine | 79 | 139.2 | 227.9 | 7.07 | 33.49 |
| 17 | (3-fluoro-5-methoxyphenyl)methanamine | 92 | 155.1 | 139.0 | 2.53 | 4.86 |
| 19 | 2-(aminomethyl)-3-methoxyphenol | 99 | 153.1 | 154.1 | 0.00 | 0.00 |
| 20 | (4-(benzyloxy)phenyl)methanamine | 80 | 213.2 | 214.0 | 0.0 | 0.0 |
| 21 | (2,3-dihydrobenzo-[b][1,4]dioxin- | 89 | 165.1 | 166.1 | 0.00 | 12.36 |
| | 5-yl)methanamine | | | | | |
| 22 | (4-methoxyphenyl)methanamine | 99 | 137.1 | 121.1 | 0.00 | 4.56 |
| 23 | (2-fluorophenyl)methanamine | 96 | 125.1 | — | 1.3 | 44.0 |
| 24 | (3,5-dimethoxyphenyl)methanamine | 95 | 167.1 | — | 0.00 | 0.75 |
| 25 | 1,4-phenylene dimethanamine | 99 | 136.2 | — | 0.00 | 0.00 |
| 26 | 1,3-phenylene dimethanamine | 99 | 136.2 | — | 0.00 | 0.00 |
| 27 | 4-(aminomethyl)benzoic acid | 97 | 151.1 | — | 0.00 | 4.06 |
| 28 | (4-methoxynaphthalen-1-yl)methanamine | 90 | 187.1 | 171.1 | 0.70 | 0.00 |
| 29 | 1-(aminomethyl)naphthalen-2-ol | 96 | 173.2 | 157.0 | 0.00 | 0.00 |
| 30 | 1-(naphthalen-1-yl)ethanamine | 99 | 171.2 | — | 11.00 | 0.00 |

TABLE 2

| | | SSAO activity | |
|---|---|---|---|
| | | (% relative to benzylamine^{a}) | |
| Entry | Compound | Human | Mouse |
| 41 | 2-phenylethanamine | 8.20 | 6.80 |
| 42 | 3-phenylpropan-1-amine | 60.30 | 44.80 |
| 43 | 2-phenoxyethanamine | 17.93 | 14.60 |
| 44 | 4-phenylbutan-1-amine | 147.63 | 61.18 |
| 45 | (4-methoxy-1H-indol-3- | 0.00 | 5.54 |
| | yl)methanamine | | |
| 46 | 2-(4-fluorophenyl)ethanamine | 16.30 | 59.80 |
| 47 | 4-(2-aminoethyl)benzene- | 0.00 | 0.00 |
| | 1,2-diol | | |
| 48 | 2-(3,4-dimethoxyphenyl)eth- | 0.00 | 3.80 |
| | anamine | | |

TABLE 3

| | | Mouse SSAO/VAP-1 | | | |
|---|---|---|---|---|---|
| | | | | | k_{cat}/ |
| | | | Vₘₐₓ | | Kₘ |
| | | | (nmol/min/ | k_{cat} | (s⁻¹ · |
| Entry | Compound | Kₘ (μM) | mg protein) | (s⁻¹) | M⁻¹) |
| 1 | Benzylamine | 27.5 ± 9.9 | 1.99 ± 0.12 | 0.104 | 3775 |
| 3 | 4-Fluorobenzylamine | 29.1 ± 4.8 | 0.83 ± 0.03 | 0.043 | 1488 |
| 41 | Phenylethylamine | 4.8 ± 13 | 0.28 ± 0.07 | 0.015 | 3043 |
| 42 | 3-Phenylpropylamine | 10 ± 1.2 | 0.32 ± 0.02 | 0.017 | 1669 |
| 44 | 4-Phenylbutylamine | 8.8 ± 2.9 | 0.66 ± 0.05 | 0.034 | 3912 |
| 46 | 2-(4-Fluorophen- | 31 ± 10 | 0.29 ± 0.02 | 0.015 | 488 |
| | yl)ethylamine | | | | |

TABLE 4

| | | Human SSAO/VAP-1 | | | |
|---|---|---|---|---|---|
| | | | | | k_{cat}/ |
| | | | Vₘₐₓ | | Kₘ |
| En- | | | (nmol/min/ | k_{cat} | (s⁻¹ · |
| try | Compound | Kₘ (μM) | mg protein) | (s⁻¹) | M⁻¹) |
| 1 | Benzylamine | 277 ± 33 | 294 ± 25 | 0.42 | 1527 |
| 3 | 4-Fluorobenzyl- | 647 ± 32 | 270 ± 63 | 0.39 | 600 |
| | amine | | | | |
| 41 | Phenylethylamine | 2918 ± 1910 | 50 ± 9 | 0.07 | 25 |
| 42 | 3-Phenylpropyl- | 1339 ± 140 | 156 ± 41 | 0.22 | 168 |
| | amine | | | | |
| 44 | 4-Phenylbutyl- | 430 ± 110 | 577 ± 30 | 0.83 | 1931 |
| | amine | | | | |
| 46 | 2-(4-Fluorophen- | 2805 ± 613 | 263 ± 16 | 0.38 | 135 |
| | yl)ethylamine | | | | |

### Example 17

### Mechanism of Action of Vanadium Arylalkylamines

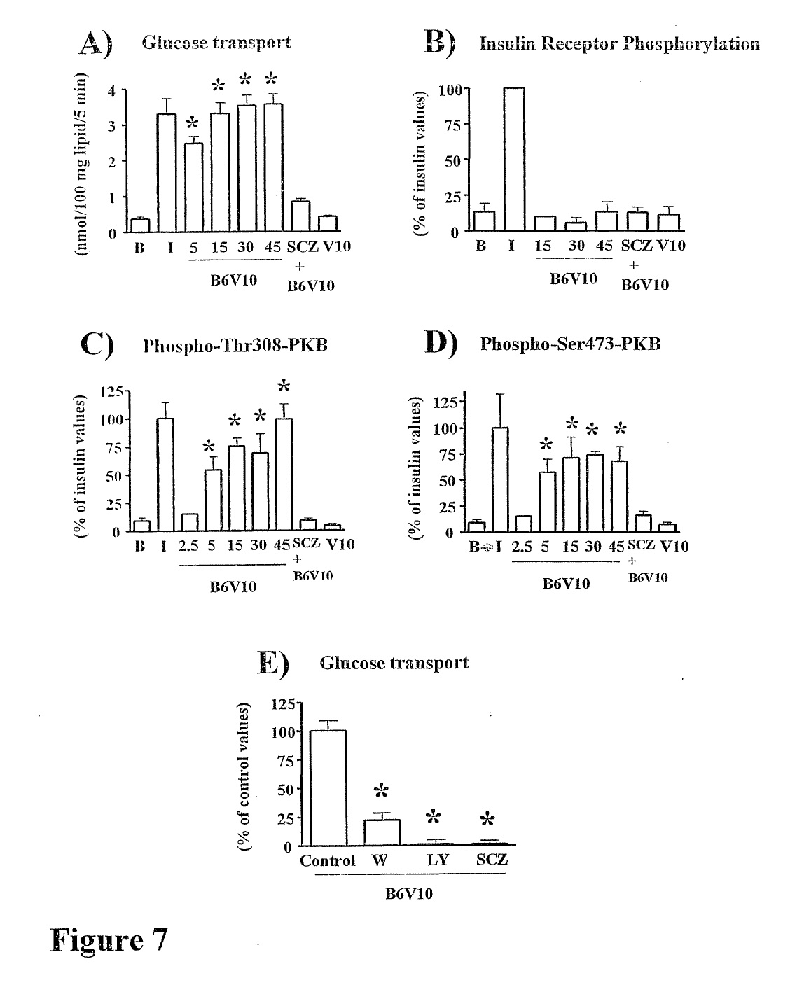

The mechanism of action of B6V10 was investigated in isolated rat adipocytes. Adipose cells from Wistar rats were incubated for different times in the presence of 25 μM hexaquis(benzylammonium) decavanadate (B6V10). Cells were also incubated in the presence of insulin (100 nM, 45 min), decavanadate (25 μM, 45 min) or semicarbazide (1 mM, 45 min). Subsequently, 2-deoxyglucose uptake (results shown in FIG. 7A), tyrosine phosphorylation of insulin receptor (FIG. 7B), phospho-Thr³⁰⁸-protein kinase B (FIG. 7C) and phospho-Ser⁴⁷³-protein kinase B (FIG. 7D) was measured. B6V10 rapidly stimulated protein kinase B as assessed by the phosphorylation of Thr⁴⁷³ and Ser⁴⁷³ in the rat insulin receptor that was detectable as early as 2.5 min after B6V10 addition (FIG. 7B). The phosphorylation of protein kinase B induced by B6V10 was parallel to activation of glucose transport (FIG. 7C and 7D). Under these conditions, tyrosine phosphorylation of insulin receptors was undetectable in adipose cells incubated with B6V10, indicating that the initial site of activation of the insulin signalling was downstream from insulin receptor.

The effects of incubation with semicarbazide or phosphatidylinositol 3-kinase inhibitors were also investigated. Adipose cells were incubated with B6V10 (25 μM, 45 min) in the absence or presence of wortmannin (2 μM, 45 min), LY294002 (10 μM, 45 min) or semicarbazide (1 mM, 45 min) and thereafter 2-deoxyglucose uptake was determined during 5 min. Activation of protein kinase B phosphorylation induced by B6V10 was blocked by semicarbazide and it was not observed by decavanadate. In addition, phosphatidylinositol 3-kinase inhibitors wortmannin and LY294002 blocked B6V10-induced glucose transport (FIG. 7E).

### Example 18

### Effectiveness of B6V10 on Glucose Tolerance In Vivo

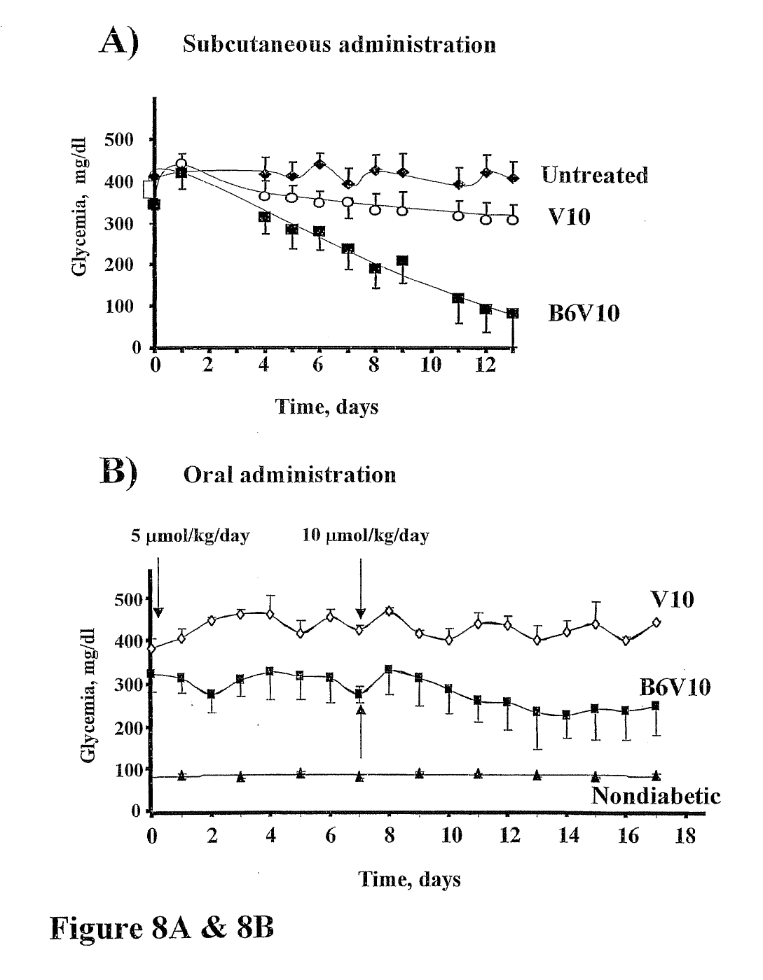

Chronic in vivo efficacy of B6V10 was evaluated in streptozotocin-induced diabetic rats and in db/db mice. Streptozotocin-induced (45 mg/kg) diabetic rats were subcutaneously treated with hexaquis(benzylammonium) decavanadate (2.5 μmol/kg) (B6V10, solid squares, FIG. 8A) or with decavanadate (2.5 μmol/kg) (V10, open circles, FIG. 8A) delivered subcutaneously by osmotic minipumps implanted in the dorsal region. Diabetic rats were also sham-operated (untreated, solid diamonds, FIG. 8A). Chronic subcutaneous administration of B6V10 for 12 days resulted in significant correction of hyperglycemia in streptozotocin-induced diabetic rats (45 mg/kg of streptozotocin) (FIG. 8A). These experiments were repeated using an oral administration protocol. Streptozotocin-induced (45 mg/kg) diabetic rats were orally treated with hexaquis(benzylammonium) decavanadate (5 μmol/kg from day 0 to day 7 and 10 μmol/kg/day from day 7 to day 17) (B6V10, solid squares, FIG. 8B) or received decavanadate (10 μmol/kg) (V10, open circles, FIG. 8B). Nondiabetic rats were also untreated (solid triangles, FIG. 8B). Daily oral administration of B6V10 for 17 days also resulted in significant correction of hyperglycemia in diabetic rats (45 mg/kg of streptozotocin) (FIG. 8B). Treatment with identical doses of decavanadate (V10) did not alter glycemia in streptozotocin-induced diabetic rats (FIGS. 8A and 8B).

### Example 19

### Insulin Replacement Activity of Vanadium Salts of Arylalkylamines

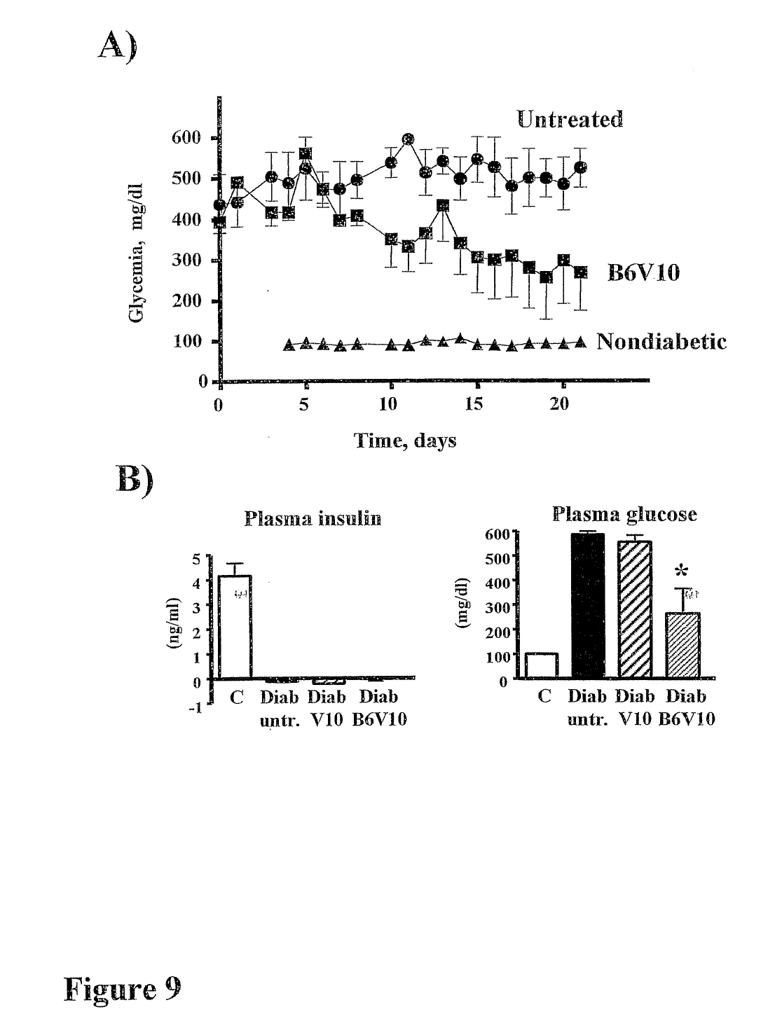

The capacity of B6V10 to exhibit antidiabetic effects in vivo in the complete absence of insulin. To this end, rats were made diabetic by the injection of a large dose of streptozotocin (100 mg/kg) that eliminates β-pancreatic insulin content. These rats showed undetectable levels of insulin in plasma (FIG. 9B). These streptozotocin-induced diabetic rats were subcutaneously treated with B6V10 (2.5 μmol/kg) (solid squares, FIG. 9A) delivered by osmotic minipumps or left untreated (solid circles, FIG. 9A). Sham-operated nondiabetic rats were also untreated (solid triangles, FIG. 9A). Diabetic rats responded to subcutaneous treatment with B6V10 by reducing glycemia (FIG. 9A). However, treatment with decavanadate did not show any change in circulating glucose (data not shown). Chronic treatment with therapeutic doses of B6V10 did not affect body weight or organ weights (data not shown).

The concentration of circulating glucose under these conditions was also assayed. After 28 days of treating diabetic rats with hexaquis(benzylammonium) decavanadate (2.5 μmol/kg) (wide striped bars, FIG. 9B), or with decavanadate (close striped bars, FIG. 9B) delivered by osmotic minipumps, plasma insulin and glucose were measured. Untreated diabetic (solid bars, FIG. 9B) or nondiabetic rats (open bars, FIG. 9B) were similarly assayed as controls. As shown in FIG. 9B, plasma glucose was reduced even without any observable change in the amount of insulin normalized in diabetic rats treated with B6V10 but not with decavanadate alone. These results indicated that B6V10 could be used to replace insulin treatment in human types 1 and 2 diabetes, based in these results in a clinically-accepted animal model of the disease

It should be understood that the foregoing disclosure emphasizes certain specific embodiments of the invention and that all modifications or alternatives equivalent thereto are within the spirit and scope of the invention as set forth in the appended claims.

The invention and the manner and process of making and using it, are now described in such full, clear, concise and exact terms as to enable any person skilled in the art to which it pertains, to make and use the same. It is to be understood that the foregoing describes preferred embodiments of the invention and that modifications may be made therein without departing from the spirit or scope of the invention as set forth in the claims. To particularly point out and distinctly claim the subject matter regarded as invention, the following claims conclude this specification.

We claim:

1. A compound having the formula: 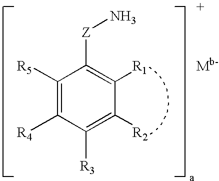
or pharmaceutically acceptable solvates or salts thereof, including hydrates, wherein: Z is —(C₁-C₆ alkoxy)- or —(C₁-C₆ alkyl)-;
R₁, R₂, R₃, R₄ and R₅ are independently H, OH, (C₁-C₆)-alkyl, (C₁-C₆)-alkoxy, NR₆R₇, (CH₂)ₚNR₈R₉, O(CH₂)_{q}Phenyl, CONR₁₀R₁₁, COR₁₂, CF₃, OCF₃, F, Cl, Br, NO₂, phenyl, CO₂R₁₂, or CH₂NHC(═NH)NH₂; or
R₁ and R₂ and the carbons to which they are attached form a benzene ring; or
R₁ and R₂ together are —O—CH₂CH₂—O—, or —O—CH₂—O—; p and q are integers from 1 to 3; R₆, R₇, R₈, R₉, R₁₀, R₁₁ and R₁₂ are radicals that are independently H, (C₁-C₄)-alkyl or phenyl; wherein a is an integer from 1 to 10, b is an integer from 1 to 10, on the condition that |a|−|b|=0. and M comprises a vanadium ion or vanadium ion complex in any possible oxoacid and polyoxometalate form at the appropriate pH.

2. A compound having the formula according to claim 1, R₁, R₂, R₃, R₄ and R₅ are radicals independently selected from H, OH, (C₁-C₆)-alkyl, (C₁-C₆)-alkoxy, O(CH₂)_{q}Phenyl, COR₁₂, CF₃, OCF₃, F, Cl, Br, NO₂, phenyl, and CO₂R₁₂.

3. A compound having the formula according to claim 2, wherein Z is —CH₂—.

4. A compound having the formula according to any of the claim 1, wherein a=6 and b=6 and M=V₁₀O₂₈.

5. A compound having the formula according to claim 4, wherein the compound is hexaquis(benzylammonium) decavanadate (V),

6. A compound having the formula to claim 4, wherein the compound is hexaquis(4-fluorobenzylamine) decavanadate (V)

7. A compound having the formula according to claim 1, wherein Z is C₃ alkyl or C₃ alkoxy.

8. A compound having the formula to the claim 7 wherein a=6 and b=6 and M=V₁₀O₂₈.

9. A compound having the formula according to claims 1, wherein Z is C₂ alkyl or C₂ alkoxy.

10. A compound having the formula of the claim 9 wherein a=6 and b=6 and M=V₁₀O₂₈.

11. A compound according to claims 1, wherein Z is C₄ alkyl or C₄ alkoxy.

12. A compound according to claim 11 wherein a=6 and b=6 and M=V₁₀O₂₈.

13. A compound according to any of the claim 1 wherein a=5 and b=5 and M=HV₁₀O₂₈.

14. A compound according to any of the claim 1 wherein a=4 and b=4 and M=H₂V₁₀O₂₈.

15. A pharmaceutical composition comprising a compound according to claim 1 and at least one pharmaceutically-acceptable excipient, diluent or adjuvant thereof.

16. A method of treating an animal having a disease, disorder or defect characterized by reduced plasma insulin or reduced or absent responsiveness to insulin, comprising administering a therapeutically-effective amount of the compound or pharmaceutically acceptable salt of claim 1 to an animal in need of such treatment.

17. A compound according to claim 1, where
R₂, R₃, and R₄ are independently H, OH, (C₁-C₆)-alkyl, (C₁-C₆)-alkoxy, NR₆R₇, (CH₂)NR₈R₉, O(CH₂)Phenyl, CONR₁₀R₁₁, COR₁₂, NO₂, phenyl, or CO₂R₁₂;
at least one of R₃, R₄ and R₅ is halogen, CF₃, or OCF₃; and
R₁ and R₅ are both H.

18. The compound according to claim 17, wherein the arylalkylamines is (4-fluorophenyl)methanamine, p-tolylmethanamine, (4-ethylphenyl)methanamine, (4-tert-butylphenyl)methanamine, (4-isopropylphenyl)methanamine, (4-(trifluoromethyl)phenyl)methanamine, (4-(trifluoromethoxy)phenyl)methanamine, (2,4-dimethoxyphenyl)methanamine, (4-butylphenyl)methanamine, 4-(aminomethyl)-2-bromophenol, 2-(aminomethyl)-6-nitrophenol, biphenyl-4-ylmethanamine, (3-fluoro-4-methylphenyl)methanamine, (3-fluoro-5-methoxyphenyl)methanamine, 2-(aminomethyl)-3-methoxyphenol, (4-(benzyloxy)phenyl)methanamine, (2,3-dihydrobenzo[b][1,4]dioxin-5-yl)methanamine, (4-methoxyphenyl)methanamine, (2-fluorophenyl)methanamine, (3,5-dimethoxyphenyl)methanamine, 1,4-phenylenedimethanamine, 1,3-phenylenedimethanamine, 4-(aminomethyl)benzoic acid, (4-methoxynaphthalen-1-yl)methanamine, 1-(aminomethyl)naphthalen-2-ol, 1-(naphthalen-1-yl)ethanamine, 2-phenylethanamine, 3-phenylpropan-1-amine, 2-phenoxyethanamine, 4-phenylbutan-1-amine, 2-(4-fluorophenyl)ethanamine, 4-(2-aminoethyl)benzene-1,2-diol or 2-(3,4-dimethoxyphenyl)ethanamine.

19. A method for preparing a compound of claim 1, comprising 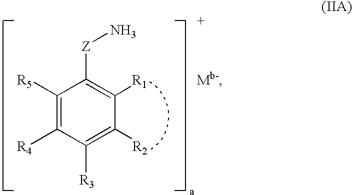
where M, a, b, Z, and R₁-R₅ are as defined in claim 1, the method comprising
a) reacting an amine of formula (II) 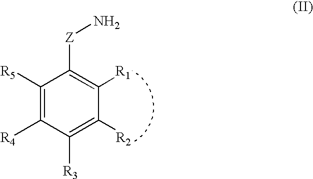
with an alkaline metal vanadate in an inert solvent at a appropriate acidity, and
b) recovering the compound of formula (IIA) from the reaction media.

20. The method of claim 19, wherein the alkaline metal vanadate is sodium vanadate.

21. The method of claim 19, wherein the solvent is water, mixtures of (C₁-C₅)— aliphatic monoalcohols with water, or mixtures of (C₃-C₇)-aliphatic ketones with water.

22. The method of claim 19, wherein the acidity corresponds to an effective pH value between about 2 and about 7.5.

23. The method of claim 19, wherein the alkaline metal vanadate is sodium vanadate, the solvent is water, mixtures of (C₁-C₅)-aliphatic monoalcohols with water, or mixtures of (C₃-C₇)-aliphatic ketones with water, and the acidity corresponds to an effective pH value between 2 and 7.5
